(19) United States
(12) Patent Application Publication
(10) Pub. No.: US 2025/0041284 A1
(43) Pub. Date: Feb. 6, 2025

(54) TREATMENT OF NEUROLOGICAL DISORDERS

(71) Applicant: Praxis Precision Medicines, Inc., Boston, MA (US)

(72) Inventors: Michael Kristopher Mathieu Kahlig, Redwood City, CA (US); Marion Wittmann, Medford, MA (US); Jonathan Novak, Boston, MA (US)

(21) Appl. No.: 18/927,451

(22) Filed: Oct. 25, 2024

Related U.S. Application Data
(63) Continuation of application No. PCT/US2023/019659, filed on Apr. 24, 2023.
(60) Provisional application No. 63/349,408, filed on Jun. 6, 2022.
(60) Provisional application No. 63/349,250, filed on Jun. 6, 2022.
(60) Provisional application No. 63/334,942, filed on Apr. 26, 2022.

(51) Int. Cl. A61K 31/444; A61P 25/08

(52) U.S. Cl. CPC A61K 31/444; A61P 25/08

(57) ABSTRACT

The present disclosure is generally directed to methods of treating a disease, disorder, or condition, e.g., a neurological disorder, a disorder associated with excessive neuronal excitability, or a disorder associated with de novo gain-of-function or loss-of-function mutations in major central nervous system sodium channel genes, such as for example, SCN1A, SCN2A, and SCN8A, using Compound 1 of the following formula:

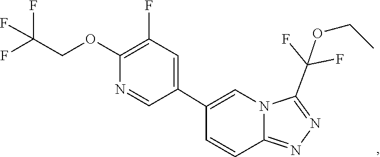 [SMILES: CCOC(F)(F)c1nnc2ccc(-c3cnc(OCC(F)(F)F)c(F)c3)cn12]

or a pharmaceutically acceptable salt thereof.

### RELATED APPLICATIONS

This application is a continuation of International Application No. PCT/US2023/019659, filed on Apr. 24, 2023; which claims the benefit of priority to U.S. Provisional Application No. 63/334,942, filed on Apr. 26, 2022; U.S. Provisional Application No. 63/349,408, filed on Jun. 6, 2022 and U.S. Provisional Application No. 63/349,250, filed on Jun. 6, 2022. The entire contents of each of the foregoing applications are hereby incorporated herein by reference.

### FIELD OF THE DISCLOSURE

The present disclosure is generally directed to methods of treating a disease, disorder, or condition, e.g., a neurological disorder, a disorder associated with excessive neuronal excitability, or a disorder associated with de novo gain-of-function or loss-of-function mutations in central nervous system sodium channel genes, such as for example, SCN1A, SCN2A, and SCN8A.

### BACKGROUND OF THE DISCLOSURE

Sodium ion (Na⁺) channels primarily open in a transient manner and are quickly inactivated, thereby generating a fast Na⁺ current to initiate the action potential. The late or persistent sodium current (I_{Na}L) is a sustained component of the fast Na⁺ current of cardiac myocytes and neurons. Many common neurological and cardiac conditions are associated with abnormal I_{Na}L enhancement, which contributes to the pathogenesis of both electrical and contractile dysfunction in mammals (see e.g., Pharmacol. Ther., 2008, 119:326-339).

Epilepsy is the fourth most common neurological disorder, affecting 3.4 million people in the United States, including 470,000 children. Epilepsy is a group of heterogeneous disorders classified into distinct syndromes by etiology, seizure type(s), and comorbidities. The most common cause of genetic epilepsy is mutations within voltage-gated sodium channel (Na_{V}) genes leading to gain-of-function and/or loss-of-function changes in channel activity. Affected patients typically present as children or neonates and have prognoses ranging from benign seizures that spontaneously remit to devastating developmental and epileptic encephalopathies (DEEs).

Na_{V} channels are an important therapeutic target for antiepileptic drugs (AEDs). Their blockade, and consequent inhibition of neuronal sodium current (I_{Na}), is ideally positioned to reduce excitability, as peak I_{Na} in the axonal initial segment and node of Ranvier is responsible for the initiation and propagation of action potentials (APs), respectively. However, the clinical utility of standard Na_{V}-targeting AEDs is limited because current agents, including cenobamate, oxcarbazepine, and phenytoin, can show severe toxicity at therapeutic doses. This toxicity includes ataxia, lethargy, vomiting, and seizures and reflects compromised physiologic neuronal function resulting from excessive peak I_{Na} inhibition or off-target (non-Na_{V}-mediated) activities. Identification of novel I_{Na} inhibitors with improved tolerability would thus represent a clinically meaningful alternative treatment option.

Physiological persistent I_{Na} is a small, subthreshold current that contributes to the amplification of synaptic responses and the enhancement of repetitive firing. Functional studies of SCN2A (encoding Na_{V}1.2) and SCN8A (encoding Na_{V}1.6) DEE variants have demonstrated small increases in persistent I_{Na} that can cause hyperexcitability, seizures, and developmental comorbidities. Current Na_{V}-targeting AEDs are predicted to inhibit both peak I_{Na} and persistent I_{Na} at or near therapeutic concentrations (high μmol/L range), with excessive peak I_{Na} inhibition compromising physiological neuronal activity. Therefore, improved selectivity for Na_{V} activity and preference in the targeting of persistent I_{Na} could meaningfully improve tolerability.

Accordingly, additional therapeutic options with improved efficacy and tolerability to treat epilepsy, to achieve seizure freedom, or both are needed.

### SUMMARY OF THE DISCLOSURE

Disclosed herein are methods of treating a disease, disorder, or condition, e.g., a neurological disorder, a disorder associated with excessive neuronal excitability and/or abnormal late sodium current, or a disorder associated with de novo gain-of-function (GoF) or loss-of-function mutations (variants) in major central nervous system sodium channel genes, such as for example, SCN1A, SCN2A, and SCN8A, by administering to a subject in need thereof a therapeutically effective amount of Compound 1 having the following formula:

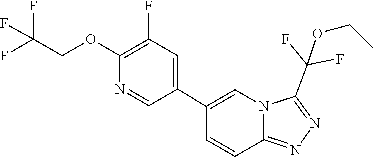 [SMILES: CCOC(F)(F)c1nnc2ccc(-c3cnc(OCC(F)(F)F)c(F)c3)cn12]

or a pharmaceutically acceptable salt thereof.

In some embodiments, the method provided involves treating a disorder associated with excessive neuronal excitability. In some embodiments, the disorder is epilepsy, an epilepsy syndrome, or an encephalopathy, such as a genetic or pediatric epilepsy or a genetic or pediatric epilepsy syndrome. In some embodiments, the disorder is focal epilepsy.

In some embodiments, the method reduces frequency of seizures experienced by the subject within 24 hours after administration of the compound or a pharmaceutically acceptable salt thereof as compared to the frequency of seizures prior to the administration.

In some embodiments, the compound of the disclosure, or a pharmaceutically acceptable salt thereof, is administered to the subject in an amount ranging from about 0.1 mg/kg to about 1 g/kg. In other embodiments, the compound or a pharmaceutically acceptable salt thereof is administered to the subject in an amount ranging from about 10 mg/kg to about 100 mg/kg, such as about 30 mg/kg.

In some embodiments, the subject is human. In some embodiments, the subject is an adult suffering from or suspected of having focal epilepsy.

In some aspects, the present disclosure provides a method of treating a condition relating to aberrant function of a sodium ion channel in a subject in need thereof, the method comprising administering to the subject Compound 1, or a pharmaceutically acceptable salt thereof, at a dose of about 1 mg to about 150 mg; wherein Compound 1 is of the following structural formula:

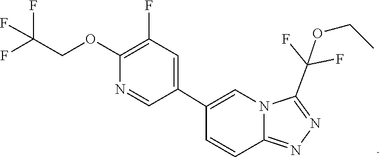 [SMILES: CCOC(F)(F)c1nnc2ccc(-c3cnc(OCC(F)(F)F)c(F)c3)cn12]

In some embodiments, the condition relating to aberrant function of a sodium ion channel is a neurological disorder. In some embodiments, the neurological disorder is a disorder associated with excessive neuronal excitability. In some embodiments, the neurological disorder is associated with one or more de novo gain-of-function or loss-of-function mutations in central nervous system sodium ion channel genes.

In some embodiments, the condition is epilepsy or an epilepsy syndrome. In some embodiments, the condition is a genetic epilepsy or a genetic epilepsy syndrome. In some embodiments, the condition is a pediatric epilepsy or a pediatric epilepsy syndrome. In some embodiments, the condition is selected from the group consisting of malignant migrating focal seizures of infancy (MMFSI), epilepsy of infancy with migrating focal seizures (EIMFS), autosomal dominant nocturnal frontal lobe epilepsy (ADNFLE), West syndrome, infantile spasms, epileptic encephalopathy, focal epilepsy, Ohtahara syndrome, developmental and epileptic encephalopathy, Lennox-Gastaut syndrome, seizures, leukodystrophy, leukoencephalopathy, intellectual disability, multifocal epilepsy, drug-resistant epilepsy, temporal lobe epilepsy and cerebellar ataxia.

In some embodiments, the condition is epileptic encephalopathy. In some embodiments, the condition is focal epilepsy.

In some embodiments, the seizures are generalized tonic clonic seizures or asymmetric tonic seizures.

In some embodiments, the subject is a human.

In some embodiments, the Compound 1 is administered at the dose of about 5 mg to about 130 mg. In some embodiments, the Compound 1 is administered at the dose of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg or about 130 mg.

In some embodiments, the administration of Compound 1 results in a reduction in the severity, number and/or frequency of seizures experienced by the subject as compared to the severity, number and/or frequency of seizures experienced by the subject prior to administration of Compound 1.

In some embodiments, the administration of Compound 1 does not result in ataxia, lethargy and vomiting in the subject.

In some aspects, the present disclosure provides a method of reducing severity, number and/or frequency of seizures in a subject in need thereof, the method comprising administering to the subject an effective amount of Compound 1 of the following structural formula:

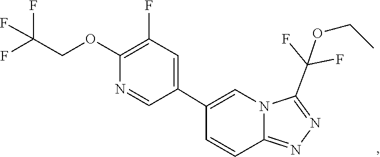 [SMILES: CCOC(F)(F)c1nnc2ccc(-c3cnc(OCC(F)(F)F)c(F)c3)cn12]

or a pharmaceutically acceptable salt thereof.

In some embodiments, the subject has a condition relating to aberrant function of a sodium ion channel. In some embodiments, the condition relating to aberrant function of a sodium ion channel is a neurological disorder. In some embodiments, the neurological disorder is a disorder associated with excessive neuronal excitability. In some embodiments, the neurological disorder is associated with one or more de novo gain-of-function or loss-of-function mutations in central nervous system sodium ion channel genes.

In some embodiments, the condition is epilepsy or an epilepsy syndrome. In some embodiments, the condition is a genetic epilepsy or a genetic epilepsy syndrome. In some embodiments, the condition is a pediatric epilepsy or a pediatric epilepsy syndrome. In some embodiments, the condition is selected from the group consisting of malignant migrating focal seizures of infancy (MMFSI), epilepsy of infancy with migrating focal seizures (EIMFS), autosomal dominant nocturnal frontal lobe epilepsy (ADNFLE), West syndrome, infantile spasms, epileptic encephalopathy, focal epilepsy, Ohtahara syndrome, developmental and epileptic encephalopathy, Lennox-Gastaut syndrome, seizures, leukodystrophy, leukoencephalopathy, intellectual disability, multifocal epilepsy, drug-resistant epilepsy, temporal lobe epilepsy and cerebellar ataxia.

In some embodiments, the the condition is epileptic encephalopathy. In some embodiments, the condition is focal epilepsy.

In some embodiments, the seizures are generalized tonic clonic seizures or asymmetric tonic seizures.

In some embodiments, the subject is a human.

In some embodiments, Compound 1 is administered at the dose of about 1 mg to about 150 mg. In some embodiments, Compound 1 is administered at the dose of about 1 mg, about 2 mg, about 3 mg, about 4 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg or about 150 mg.

In some embodiments, the administration of Compound 1 does not result in ataxia, lethargy and vomiting in the subject.

In some aspects, the present disclosure provides a method of preferentially inhibiting persistent sodium current (I_{Na}) over peak sodium current (I_{Na}) in a neuron, the method comprising contacting said neuron with an effective amount of Compound 1 of the following structural formula:

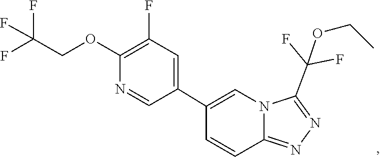 [SMILES: CCOC(F)(F)c1nnc2ccc(-c3cnc(OCC(F)(F)F)c(F)c3)cn12]

or a pharmaceutically acceptable salt thereof.

In some embodiments, the neuron is in a subject. In some embodiments, the subject has a condition relating to aberrant function of a sodium ion channel. In some embodiments, the condition relating to aberrant function of a sodium ion channel is a neurological disorder. In some embodiments, the neurological disorder is a disorder associated with excessive neuronal excitability. In some embodiments, the neurological disorder is associated with one or more de novo gain-of-function or loss-of-function mutations in central nervous system sodium ion channel genes.

In some embodiments, the condition is epilepsy or an epilepsy syndrome. In some embodiments, the condition is a genetic epilepsy or a genetic epilepsy syndrome. In some embodiments, the condition is a pediatric epilepsy or a pediatric epilepsy syndrome. In some embodiments, the condition is selected from the group consisting of malignant migrating focal seizures of infancy (MMFSI), epilepsy of infancy with migrating focal seizures (EIMFS), autosomal dominant nocturnal frontal lobe epilepsy (ADNFLE), West syndrome, infantile spasms, epileptic encephalopathy, focal epilepsy, Ohtahara syndrome, developmental and epileptic encephalopathy, Lennox-Gastaut syndrome, seizures, leukodystrophy, leukoencephalopathy, intellectual disability, multifocal epilepsy, drug-resistant epilepsy, temporal lobe epilepsy and cerebellar ataxia.

In some embodiments, the condition is epileptic encephalopathy. In some embodiments, the condition is focal epilepsy.

In some embodiments, the seizures are generalized tonic clonic seizures or asymmetric tonic seizures.

In some embodiments, the subject is a human.

### BRIEF DESCRIPTION OF THE DRAWINGS

The accompanying drawings, which are incorporated in and constitute a part of this specification, illustrate certain embodiments, and together with the written description, serve to explain certain principles of the methods and devices disclosed herein.

FIGS. 1A-1D depict the effect of Compound 1 (FIG. 1A), Reference Compound (FIG. 1B), lamotrigine (LTG) (FIG. 1C), and carbamazepine (CBZ) (FIG. 1D) on human Na_{V}1.6 channel using the PatchXpress® (Molecular Devices) Electrophysiology platform. ● Persistent I_{Na} Inhibition; Δ Peak I_{Na}, UDV-10 Hz (Disease-State Dependence) Inhibition; ⋄ Peak I_{Na}, Tonic Block Inhibition.

FIG. 2 depicts the effect of Compound 1 on maximal electroshock induced seizure (MES) in male CD-1 mice. CD-1 mice: n=8/group veh, n=10/group Compound 1; ANOVA/Dunnett (Compound 1); **p<0.01 vs. Veh.

FIG. 3 depicts the effect of Compound 1 on spontaneous locomotor activity (sLMA) in male CD-1 mice. CD-1 mice: n=10/group; ANOVA/Dunnett; **p<0.01 vs. Veh.

FIG. 4 depicts the range of calculated free plasma concentrations of Compound 1, Reference Compound, CBZ, and LTG associated with anticonvulsant effects (efficacious exposure) and reductions in locomotor activity (intolerable exposure). Protective indexes for each molecule are shown.

FIG. 5 depicts a pharmacokinetics (PK) modeling of Compound 1 and Reference Compound based on a 90-mg single dose. Preclinical simulation of human PK recapitulates Reference Compound clinical data.

FIG. 6A shows the result of assessment of I_{Na} block using an assay for tonic block in HEK cells.

FIG. 6B shows the result of assessment of I_{Na} block using an assay for use-dependent block in HEK cells.

FIG. 6C shows the result of assessment of I_{Na} block using an assay for voltage-dependent block in HEK cells. Peak I_{Na} was measured at the beginning of the voltage step. FIGS. 6A-6C indicate that Compound 1 exhibited enhanced activity-dependent block which has been suggested to convey beneficial activity during periods of hyperexcitability.

FIG. 7A is a graph showing percent inhibition of hNa_{V}1.6 as a function of the concentration of Compound 1.

FIG. 7B is a graph showing percent inhibition of hNa_{V}1.6 as a function of the concentration of carbamazepine.

FIG. 7C is a graph showing percent inhibition of hNa_{V}1.6 as a function of the concentration of lamotrigine.

FIG. 8A shows the results of Compound 1-induced reduction in ATX-II evoked hNa_{V}1.6 persistent I_{Na}. Voltage protocol included as panel inset; pharmacology measured at arrow.

FIG. 8B is a graph showing percent inhibition of hNa_{V}1.6 as a function of the concentration for Compound 1 and standard Na_{V}-targeting ASMs (lamotrigine, phenytoin, carbamazepine, cenbamate, lacosamide and valproic acid). Points represent mean±SEM.

FIG. 8C is a graph showing percent inhibition of various Na_{V} isoforms and orthologs as a function of the concentration of Compound 1. Points represent mean±SEM.

FIG. 9A shows development of inhibition (apparent binding) for 3 μM Compound 1 and control.

FIG. 9B is a graph showing normalized I_{Na} as a function of inactivation time and illustrates the development of inactivation in the absence and presence of 3 μM Compound 1.

FIG. 9C is a graph showing normalized I_{Na} as a function of inactivation time, and illustrates the development of inhibition in the presence of Compound 1 at the concentrations of 0.3 μM, 1 μM, 3 μM, 4.5 μM, and 6 μM.

FIG. 9D is a graph showing inhibition rate as a fuction of the concentration of Compound 1 and illustrates that the apparent K_{ON} for Compound 1 is 4.2 s⁻¹*μM⁻¹.

FIG. 9E shows recovering from inhibition (apparent unbinding) for 3 μM Compound 1 and control.

FIG. 9F is a graph showing normalized I_{Na} as a function of recovery time and illustrates recovery from inactivation in the absence and presence of 3 μM Compound 1.

FIG. 9G is a graph showing normalized I_{Na} as a function of recovery time and illustrates recovery from inhibition (normalized to remove compound independent inactivation) and that K_{OFF} for Compound 1 is 1.7 s⁻¹.

FIG. 9H is a graph showing binding K_{ON} and unbinding K_{OFF} for Compound 1 and standard-of-care Nav-targeting ASMs.

FIG. 10A is a graph showing total distance travelled in the sLMA assay plotted as a percent of control vs. administered dose of Compound 1.

FIG. 10B is a graph showing total distance travelled in the sLMA assay plotted as a percent of control vs. the concentration of Compound 1 in plasma.

FIG. 10C is a graph showing total distance travelled in the sLMA assay plotted as a percent of control vs. the concentration of Compound 1 in the brain. Data are expressed as mean±SEM, with n=20 per group.

FIG. 11A is a graph showing protection from MES-induced tonic hindlimb extension as a function of the administered dose of Compound 1.

FIG. 11B is a graph showing protection from MES-induced tonic hindlimb extension as a function of the concentration of Compound 1 in plasma.

FIG. 11C is a graph showing protection from MES-induced tonic hindlimb extension as a function of the concentration of Compound 1 in the brain. Data are expressed as mean±SEM, with n=30 per group. The curves represent a fit to a four-parameter log function and EC₅₀ values are included in the figures.

FIG. 11D is a graph showing protection from MES-induced tonic hindlimb extension for Compound 1, carbamazepine, cenobamate, lamotrigine and XEN1101 as a function of the administered dose. The curves represent a fit to four-parameter log function and error bars have been removed for clarity.

FIG. 12A is a graph showing percent protection from PTZ-induced clonic seizures as a function of the administered dose of Compound 1.

FIG. 12B is a graph showing percent protection from PTZ-induced clonic seizures as a function of the concentration of Compound 1 in plasma.

FIG. 12C is a graph showing percent protection from PTZ-induced clonic seizures as a function of the concentration of Compound 1 in the brain. Data are expressed as mean±SEM, with n=10-20 per group. The curves represent a fit to a four-parameter log function and EC₅₀ values are included in the figures.

FIG. 13A is a bar graph showing seizure score in the 6-Hz acute seizure model at stimulation current of 32 mA as a function of the administered dose of Compound 1.

FIG. 13B is a bar graph showing seizure score in the 6-Hz acute seizure model at stimulation current of 44 mA as a function of the administered dose of Compound 1.

FIG. 13C is a graph showing percent protection from seizures induced by 6-Hz at stimulation currents of 32 mA (solid symbols, solid line) and 44 mA (open symbols, dashed line) as a function of the concentration of Compound 1 in plasma.

FIG. 13D is a graph showing percent protection from seizures induced by 6-Hz at stimulation currents of 32 mA (solid symbols, solid line) and 44 mA (open symbols, dashed line) as a function of the concentration of Compound 1 in the brain. Data are expressed as mean±SEM, with n=10 per group. The curves represent a fit to a four-parameter log function and EC₅₀ values as shown in the table below.

FIG. 14A is an illustration of the dosing scheme for Part A (single ascending dose) of a phase 1 clinical trial to evaluate the safety, tolerability, pharmacokinetics and pharmacodynamics of single and multiple ascending doses of Compound 1 in healthy volunteers.

FIG. 14B is an illustration of the dosing scheme for Part B (multiple ascending dose) of a phase 1 clinical trial to evaluate the safety, tolerability, pharmacokinetics and pharmacodynamics of single and multiple ascending doses of Compound 1 in healthy volunteers.

FIG. 14C is an illustration of the dosing scheme for Part C (optional food effect evaluation) of a phase 1 clinical trial to evaluate the safety, tolerability, pharmacokinetics and pharmacodynamics of single and multiple ascending doses of Compound 1 in healthy volunteers.

FIG. 15 is an illustration of the dosing scheme for a phase 2 trial to evaluate the photoparoxysmal electroencephalogram response, safety, tolerability, and pharmacokinetics of Compound 1 in participants with epilepsy and a photoparoxysmal electroencephalogram response to intermittent photic stimulation.

### DETAILED DESCRIPTION THE DISCLOSURE

Reference will now be made in detail to various exemplary embodiments, examples of which are illustrated in the accompanying drawings. It is to be understood that the following detailed description is provided to give the reader a fuller understanding of certain embodiments, features, and details of aspects of the disclosure, and should not be interpreted as a limitation of the scope of the disclosure.

### Definitions

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

The term “about” is used herein to mean within the typical ranges of tolerances in the art. For example, “about” can be understood as about 2 standard deviations from the mean. According to certain embodiments, when referring to a measurable value such as an amount and the like, “about” is meant to encompass variations of ±20%, ±10%, ±5%, ±1%, ±0.9%, ±0.8%, ±0.7%, ±0.6%, ±0.5%, ±0.4%, ±0.3%, ±0.2% or ±0.1% from the specified value as such variations are appropriate to perform the disclosed methods and/or to make and use the disclosed devices. When “about” is present before a series of numbers or a range, it is understood that “about” can modify each of the numbers in the series or range.

The terms “administer,” “administering” or “administration” as used herein refer to either directly administering a compound or pharmaceutically acceptable salt or ester of the compound or a composition comprising the compound or pharmaceutically acceptable salt or ester of the compound to a subject.

The term “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

The term “at least” prior to a number or series of numbers (e.g., “at least two”) is understood to include the number adjacent to the term “at least,” and all subsequent numbers or integers that could logically be included, as clear from context. When “at least” is present before a series of numbers or a range, it is understood that “at least” can modify each of the numbers in the series or range.

The terms “disease,” “disorder,” and “condition” are used interchangeably herein.

As used herein, the term “in some embodiments” refers to embodiments of all aspects of the disclosure, unless the context clearly indicates otherwise.

As used herein, the “effective amount” of a compound refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the age, health, and condition of the subject. An effective amount encompasses therapeutic and prophylactic treatment.

As used herein, “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

As used herein, “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, a “subject” to which administration is contemplated includes, but is not limited to, humans (i.e., a male or female of any age group, e.g., a fetus, a pediatric subject, such as an infant, a child, or an adolescent) or an adult subject (e.g., a young adult, a middle-aged adult or a senior adult) and/or a non-human animal, e.g., a mammal such as a primate (e.g., a cynomolgus monkeys or a rhesus monkeys), a cattle, a pig, a horse, a sheep, a goat, a rodent, a cat, or a dog. In certain embodiments, the subject is a human. In certain embodiments, the subject is a non-human animal.

As used herein, the terms “treat”, “treatment” or “treating” a condition or a disorder, e.g., epilepsy or an epilepsy syndrome, such as focal epilepsy, in a subject in need thereof includes achieving, partially, substantially or completely, one or more of the following: ameliorating, improving or achieving a reduction in the severity of at least one symptom of indicator associated with the condition or disorder; or arresting the progression or worsening of the condition or disorder.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

The embodiments disclosed herein are not intended to be limited in any manner by the above exemplary listing of chemical groups and substituents. Those skilled in the art will recognize that several embodiments are possible within the scope and spirit of the present disclosure. The following description illustrates the disclosure and, of course, should not be construed in any way as limiting the scope of the inventions described herein.

### Compounds and Compositions

In one aspect, provided herein is Compound 1 having the following formula:

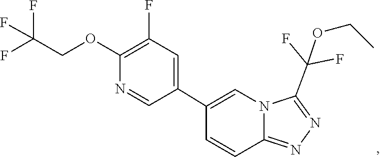 [SMILES: CCOC(F)(F)c1nnc2ccc(-c3cnc(OCC(F)(F)F)c(F)c3)cn12]

or a pharmaceutically acceptable salt thereof.

In some embodiments, Compound 1 is in a crystalline form. In some embodiments, the crystalline form may be characterized by an X-ray powder diffraction pattern comprising X-ray powder diffraction peaks at the following diffraction angles (° 2θ): 12.6±0.2, 15.8±0.2, and 18.6±0.2. In some embodiments, the crystalline form may be characterized by an X-ray powder diffraction pattern comprising X-ray powder diffraction peaks at the following diffraction angles (° 2θ): 10.7±0.2, 12.3±0.2, 12.6±0.2, 15.8±0.2, 18.6±0.2, and 22.6±0.2. In some embodiments, the crystalline form may be characterized by an X-ray powder diffraction pattern comprising X-ray powder diffraction peaks at the following diffraction angles (° 2θ): 10.7±0.2, 12.3±0.2, 12.6±0.2, 14.9±0.2, 15.8±0.2, 16.6±0.2, 16.8±0.2, 18.6±0.2, 21.0±0.2 and 22.6±0.2. The crystalline form of Compound 1 is described, e.g., in WO 2019/232209, the entire contents of which are hereby incorporated herein by reference.

Compound 1 or a pharmaceutically acceptable salt thereof described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomers. Isomers can be isolated from mixtures by methods known to those skilled in the art, including chiral high pressure liquid chromatography (HPLC) and the formation and crystallization of chiral salts; preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). Embodiments disclosed herein additionally encompass compounds described herein as individual isomers substantially free of other isomers, and alternatively, as mixtures of various isomers.

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than about 75% by weight, such as more than about 80% by weight, more than about 85% by weight, more than about 90% by weight, more than about 91% by weight, more than about 92% by weight, more than about 93% by weight, more than about 94% by weight, more than about 95% by weight, more than about 96% by weight, more than about 97% by weight, more than about 98% by weight, more than about 98.5% by weight, more than about 99% by weight, more than about 99.2% by weight, more than about 99.5% by weight, more than about 99.6% by weight, more than about 99.7% by weight, more than about 99.8% by weight, or more than about 99.9% by weight, of the enantiomer. In certain embodiments, the weights are based upon total weight of all enantiomers or stereoisomers of the compound.

In certain aspects, provided are compositions comprising Compound 1 or a pharmaceutically acceptable salt thereof described herein. In some embodiments, the composition is a pharmaceutical composition comprising Compound 1 or a pharmaceutically acceptable salt thereof described herein and a pharmaceutically acceptable carrier.

In some embodiments, an enantiomerically pure compound can be present in the compositions with other active or inactive ingredients. For example, a pharmaceutical composition comprising enantiomerically pure R-compound can comprise, for example, about 90% excipient and about 10% enantiomerically pure R-compound. In certain embodiments, the enantiomerically pure R-compound in such compositions can, for example, comprise at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In certain embodiments, the enantiomerically pure S-compound in such compositions can, for example, comprise at least about 95% by weight S-compound and at most about 5% by weight R-compound, by total weight of the compound. In certain embodiments, the active ingredient can be formulated with little or no excipient or carrier.

Compounds described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C. O may be in any isotopic form, including ¹⁶O and ¹⁸O, and F may be in any isotopic form, including ¹⁸F and ¹⁹F.

### Methods of Treatment

Compounds and compositions described herein are generally useful for modulating the activity of sodium channels and are useful in treating conditions relating to aberrant function of a sodium channel ion channel, e.g., abnormal late sodium (I_{Na}L) current. In some embodiments, a compound provided by the disclosure is effective in the treatment of epilepsy or an epilepsy syndrome. A provided compound, pharmaceutically acceptable salt thereof, or composition comprising the same may also modulate all sodium ion channels, or may be specific to only one or a plurality of sodium ion channels, e.g., Na_{V}1.1, 1.2, 1.3, 1.4, 1.5, 1.6, 1.7, 1.8, and/or 1.9. In some embodiments, a compound of the disclosure has specificity to sodium ion channel Na_{V}1.6.

In typical embodiments, the disclosure is intended to encompass the compounds disclosed herein, and the pharmaceutically acceptable salts, pharmaceutically acceptable esters, tautomeric forms, polymorphs, and prodrugs of such compounds. In some embodiments, the disclosure includes a pharmaceutically acceptable addition salt, a pharmaceutically acceptable ester, a solvate (e.g., hydrate) of an addition salt, a tautomeric form, a polymorph, an enantiomer, a mixture of enantiomers, a stereoisomer or mixture of stereoisomers (pure or as a racemic or non-racemic mixture) of Compound 1.

Compound 1 or a pharmaceutically acceptable salt thereof disclosed herein, and compositions described herein, can be used to treat a neurological disorder, a disorder associated with excessive neuronal excitability, or a disorder associated with de novo gain-of-function or loss-of-function mutations in central nervous system sodium channel genes, such as for example, SCN1A, SCN2A, and SCN8A.

In some aspects, provided are methods of treating a neurological disorder, a disorder associated with excessive neuronal excitability, or a disorder associated with de novo gain-of-function or loss-of-function mutations in the major central nervous system sodium channel genes, comprising administering to a subject in need thereof an effective amount of Compound 1 or a pharmaceutically acceptable salt thereof disclosed herein, or pharmaceutical compositions comprising the same.

Exemplary diseases, disorders, or conditions include epilepsy and other encephalopathies (e.g., malignant migrating focal seizures of infancy (MMFSI) or epilepsy of infancy with migrating focal seizures (EIMFS), autosomal dominant nocturnal frontal lobe epilepsy (ADNFLE), West syndrome, infantile spasms, epileptic encephalopathy, developmental and epileptic encephalopathy (DEE), early infantile epileptic encephalopathy (EIEE), generalized epilepsy, focal epilepsy, multifocal epilepsy, temporal lobe epilepsy, Ohtahara syndrome, early myoclonic encephalopathy, Lennox-Gastaut syndrome), and drug resistant epilepsy, seizures (e.g., frontal lobe seizures, generalized tonic clonic seizures, asymmetric tonic seizures, focal seizures).

Epilepsy is a CNS disorder in which nerve cell activity in the brain becomes disrupted, causing seizures or periods of unusual behavior, sensations and sometimes loss of consciousness. Seizure symptoms will vary widely, from a simple blank stare for a few seconds to repeated twitching of their arms or legs during a seizure. Epilepsy may involve a generalized seizure or a partial or focal seizure. All areas of the brain are involved in a generalized seizure. A person experiencing a generalized seizure may cry out or make some sound, stiffen for several seconds to a minute a then have rhythmic movements of the arms and legs. The eyes are generally open, the person may appear not to be breathing and may actually turn blue. The return to consciousness is gradual and the person may be confused from minutes to hours. There are six main types of generalized seizures: tonic-clonic, tonic, clonic, myoclonic, absence, and atonic seizures. In a partial or focal seizure, only part of the brain is involved, so only part of the body is affected. Depending on the part of the brain having abnormal electrical activity, symptoms may vary.

Epilepsy, as described herein, includes a generalized, partial, complex partial, tonic clonic, clonic, tonic, refractory seizures, status epilepticus, absence seizures, febrile seizures, or temporal lobe epilepsy.

In some embodiments, the epilepsy syndrome is early-onset DEE. In certain embodiments, the epilepsy syndrome is DEE, including, for example, Ohtahara Syndrome; epilepsy with migrating focal seizures of infancy (EIMFS); infantile and childhood DEE, for example West Syndrome and Lennon-Gastaut Syndrome; Dravet Syndrome; Idiopathic/Generic Generalized Epilepsies (IGE/GGE); Temporal Lobe Epilepsy; Myoclonic Astatic Epilepsy (MAE); Migrating Partial Epilepsy of Infancy (MMPSI); and familial hemiplegic migraines, with or without epilepsy. In certain embodiments, the epilepsy syndrome is late seizure onset epileptic encephalopathy.

In some embodiments, the epilepsy syndrome is focal epilepsy, including, for example, idiopathic location-related epilepsies (ILRE), frontal lobe epilepsy, temporal lobe epilepsy, parietal lobe epilepsy and occipital lobe epilepsy. In some embodiments, the epilepsy syndrome is adult focal epilepsy.

In some embodiments, Compound 1 or a pharmaceutically acceptable salt thereof disclosed herein, or pharmaceutical compositions comprising the same may be used in the treatment of epilepsy syndromes. Severe syndromes with diffuse brain dysfunction caused, at least partly, by some aspect of epilepsy, are also referred to as epileptic encephalopathies. These are associated with frequent seizures that are resistant to treatment and severe cognitive dysfunction, for instance West syndrome. In some embodiments, the epilepsy syndrome comprises an epileptic encephalopathy, such as Dravet syndrome, Angelman syndrome, CDKL5 disorder, frontal lobe epilepsy, infantile spasms, West's syndrome, Juvenile Myoclonic Epilepsy, Landau-Kleffner syndrome, Lennox-Gastaut syndrome, Ohtahara syndrome, PCDH19 epilepsy, or Glut1 deficiency.

In some embodiments, Compound 1 or a pharmaceutically acceptable salt thereof disclosed herein, or pharmaceutical compositions comprising the same may be used in the treatment of focal epilepsy, including, for example, idiopathic location-related epilepsies (ILRE), frontal lobe epilepsy, temporal lobe epilepsy, parietal lobe epilepsy and occipital lobe epilepsy. In some embodiments, the epilepsy syndrome is adult focal epilepsy. Focal epilepsy is a neurological condition in which the predominant symptom is recurring seizures that affect one hemisphere (half) of the brain. Thus, focal epilepsies are generally characterized by seizures arising from a specific part (lobe) of the brain.

In some embodiments, the epilepsy or epilepsy syndrome is a genetic epilepsy or a genetic epilepsy syndrome. In some embodiments, epilepsy or an epilepsy syndrome comprises epileptic encephalopathy, epileptic encephalopathy with SCN1A, SCN2A, SCN8A mutations, early infantile epileptic encephalopathy, Dravet syndrome, Dravet syndrome with SCN1A mutation, generalized epilepsy with febrile seizures, intractable childhood epilepsy with generalized tonic-clonic seizures, infantile spasms, benign familial neonatal-infantile seizures, SCN2A epileptic encephalopathy, focal epilepsy with SCN3A mutation, cryptogenic pediatric partial epilepsy with SCN3A mutation, SCN8A epileptic encephalopathy, sudden unexpected death in epilepsy (SUDEP), Rasmussen encephalitis, malignant migrating partial seizures of infancy, autosomal dominant nocturnal frontal lobe epilepsy, KCNQ2 epileptic encephalopathy, or KCNT1 epileptic encephalopathy.

In some embodiments, the methods described herein further comprise identifying a subject having epilepsy or an epilepsy syndrome (e.g., epileptic encephalopathy, epileptic encephalopathy with SCN1A, SCN2A, SCN8A mutations, early infantile epileptic encephalopathy, Dravet syndrome, Dravet syndrome with SCN1A mutation, generalized Epilepsy with febrile seizures, intractable childhood epilepsy with generalized tonic-clonic seizures, infantile spasms, benign familial neonatal-infantile seizures, SCN2A epileptic encephalopathy, focal epilepsy with SCN3A mutation, cryptogenic pediatric partial epilepsy with SCN3A mutation, SCN8A epileptic encephalopathy, sudden unexpected death in epilepsy (SUDEP), Rasmussen encephalitis, malignant migrating partial seizures of infancy, autosomal dominant nocturnal frontal lobe epilepsy, KCNQ2 epileptic encephalopathy, or KCNT1 epileptic encephalopathy) prior to administration of Compound 1 or a pharmaceutically acceptable salt thereof disclosed herein, or pharmaceutical compositions comprising the same.

In one aspect, the disclosure features a method of treating epilepsy or an epilepsy syndrome (e.g., epileptic encephalopathy, epileptic encephalopathy with SCN1A, SCN2A, SCN8A mutations, early infantile epileptic encephalopathy, developmental and epileptic encephalopathy, Dravet syndrome, Dravet syndrome with SCN1A mutation, generalized epilepsy with febrile seizures, intractable childhood epilepsy with generalized tonic-clonic seizures, infantile spasms, benign familial neonatal-infantile seizures, SCN2A epileptic encephalopathy, focal epilepsy with SCN3A mutation, cryptogenic pediatric partial epilepsy with SCN3A mutation, SCN8A epileptic encephalopathy, sudden unexpected death in epilepsy (SUDEP), Rasmussen encephalitis, malignant migrating partial seizures of infancy, autosomal dominant nocturnal frontal lobe epilepsy, KCNQ2 epileptic encephalopathy, or KCNT1 epileptic encephalopathy) comprising administering to a subject in need thereof Compound 1 or a pharmaceutically acceptable salt thereof disclosed herein, or pharmaceutical compositions comprising the same.

Compound 1 or a pharmaceutically acceptable salt thereof disclosed herein, or pharmaceutical compositions comprising the same may also be used to treat an epileptic encephalopathy, wherein the subject has a mutation in one or more of the following genes: ALDH7A1, ALG13, ARHGEF9, ARX, ASAH1, CACNA1G, CDKL5, CHD2, CHRNA2, CHRNA4, CHRNB2, CLN8, CNTNAP2, CPA6, CSTB, DEPDC5, DNM1, EEFIA2, EPM2A, EPM2B, GABRA1, GABRA2, GABRB3, GABRG2, GNAO1, GOSR2, GRIK1, GRIN1, GRIN2A, GRIN2B, HCN1, IER3IP1, KCN1A, KCNA2, KCNB1, KCNC1, KCNMA1, KCNN2, KCNQ2, KCNQ3, KCNT1, KCTD7, LGI1, MEF2C, NHLRC1, PCDH7, PCDH19, PLCB1, PNKP, PNPO, PRICKLE1, PRICKLE2, PRRT2, RELN, SCARB2, SCN1A, SCN1B, SCN2A, SCN8A, SCN9A, SHANK3, SIAT9, SIK1, SLC13A5, SLC25A22, SLC2A1, SLC35A2, SLC6A1, SNIP1, SPTAN1, SRPX2, ST3GAL3, STRADA, STX1B, STXBP1, SYN1, SYNGAP1, SZT2, TBC1D24, TRIM3, UNC79, and WWOX.

In some embodiments, the methods described herein further comprise identifying a subject having a mutation in one or more of ALDH7A1, ALG13, ARHGEF9, ARX, ASAH1, CACNA1G, CDKL5, CHD2, CHRNA2, CHRNA4, CHRNB2, CLN8, CNTNAP2, CPA6, CSTB, DEPDC5, DNM1, EEF1A2, EPM2A, EPM2B, GABRA1, GABRA2, GABRB3, GABRG2, GNAO1, GOSR2, GRIK1, GRIN1, GRIN2A, GRIN2B, HCN1, IER3IP1, KCN1A, KCNA2, KCNB1, KCNC1, KCNMA1, KCNN2, KCNQ2, KCNQ3, KCNT1, KCTD7, LGI1, MEF2C, NHLRC1, PCDH7, PCDH19, PLCB1, PNKP, PNPO, PRICKLE1, PRICKLE2, PRRT2, RELN, SCARB2, SCN1A, SCN1B, SCN2A, SCN8A, SCN9A, SHANK3, SIAT9, SIK1, SLC13A5, SLC25A22, SLC2A1, SLC35A2, SLC6A1, SNIP1, SPTAN1, SRPX2, ST3GAL3, STRADA, STX1B, STXBP1, SYN1, SYNGAP1, SZT2, TBC1D24, TRIM3, UNC79, and WWOX prior to administration of Compound 1 or a pharmaceutically acceptable salt thereof disclosed herein, or pharmaceutical compositions comprising the same.

Compound 1 or a pharmaceutically acceptable salt thereof disclosed herein, or pharmaceutical compositions comprising the same may also be used in methods for ameliorating at least one symptom or hallmark of epilepsy or epilepsy syndrome, including, for example, early-onset DEE, in a subject in need thereof. In certain embodiments, the symptom or hallmark includes one or more of seizures, hypotonia, sensory issues, such as sensory integration disorders, motor dysfunctions, intellectual and cognitive dysfunctions, movement and balance dysfunctions, such as choreoathetosis, dystonia, and ataxia, anxiety, sensory issues, urinary retention problems, irritability, behavior issues, visual dysfunctions, delayed language and speech, gastrointestinal disorders (for example, gastroesophageal reflux, diarrhea, constipation, dysmotility, and the like), neurodevelopmental delays, sleep problems, sudden unexpected death in epilepsy (SUDEP), motor development delays, delayed social milestones, repetitive actions, uncoordinated oral movements. In certain embodiments, the seizures include focal, clonic, tonic, and generalized tonic and clonic seizures, prolonged seizures (often lasting longer than 10 minutes), and frequent seizures (for example, convulsive, myoclonic, absence, focal, obtundation status, and tonic seizures).

In one aspect, the disclosure provides a method of ameliorating at least one symptom or hallmark of epilepsy or epilepsy syndrome, including, for example, focal epilepsy, the method comprising administering to a subject in need thereof Compound 1 or a pharmaceutically acceptable salt thereof disclosed herein, or pharmaceutical compositions comprising the same. In certain embodiments, the symptom or hallmark includes one or more of seizures, hypotonia, sensory issues, such as sensory integration disorders, motor dysfunctions, intellectual and cognitive dysfunctions, movement and balance dysfunctions, such as choreoathetosis, dystonia, and ataxia, anxiety, sensory issues, urinary retention problems, irritability, behavior issues, visual dysfunctions, delayed language and speech, gastrointestinal disorders (for example, gastroesophageal reflux, diarrhea, constipation, dysmotility, and the like), neurodevelopmental delays, sleep problems, sudden unexpected death in epilepsy (SUDEP), motor development delays, delayed social milestones, repetitive actions, uncoordinated oral movements. In certain embodiments, the seizures include focal, clonic, tonic, and generalized tonic and clonic seizures, prolonged seizures (often lasting longer than 10 minutes), and frequent seizures (for example, convulsive, myoclonic, absence, focal, obtundation status, and tonic seizures).

In some aspects, the present disclosure provides a method of reducing severity, number and/or frequency of seizures in a subject in need thereof that comprises administering to the subject an effective amount of Compound 1, or a pharmaceutically acceptable salt thereof. In some embodiments, the subject has epilepsy or an epilepsy syndrome. In some embodiments, the subject has focal epilepsy.

In some embodiments, provided herein is a method of treating a neurological disorder or a psychiatric disorder, wherein the method comprises administering to a subject in need thereof Compound 1 or a pharmaceutically acceptable salt thereof disclosed herein, or pharmaceutical compositions comprising the same.

In one aspect, provided herein is a method of preferentially inhibiting persistent sodium current (I_{Na}) over peak sodium current (I_{Na}) in a neuron, said method comprising contacting said neuron with an effective amount of Compound 1. As described in Example 7 herein, Compound 1 exhibited preference for inhibiting persistent I_{Na} over peak sodium current I_{Na}. Specifically, Compound 1 inhibited persistent I_{Na} in hNa_{V}1.6, and exhibited preference for the inhibition of persistent I_{Na} as compared to the inhibition of peak I_{Na}, with the ratio of peak I_{Na} to persistent I_{Na} being 68. In contrast, standard-of-care anti-epileptic drugs (AEDs) exhibited less preference for the inhibition of persistent I_{Na} as compared to the inhibition of peak I_{Na}, with the ratio of peak I_{Na} to persistent I_{Na} being less than 68. Specifically, the ratio of peak I_{Na} to persistent I_{Na} for various AEDs was 24 (cenobamate), 30 (carbamazepine), 8 (oxcarbazepine) and 16 (lamotrigine). As described herein, preferential inhibition of persistent I_{Na} over peak I_{Na} may be associated with improved tolerability of Compound 1.

In various embodiments, a method of treatment provided herein yields benefits over any other therapy, such as for example benefits over a standard-of-care therapy. In some embodiments, a method herein provides increased selectivity for a hyperexcitable neuronal state, spares normal neuronal function, or provides a wider therapeutic window than another therapy.

In any of the methods disclosed herein, Compound 1 or a pharmaceutically acceptable salt thereof disclosed herein, or pharmaceutical compositions comprising the same, is administered to the subject in an effective amount, which is an amount sufficient to elicit the desired biological response. An effective amount encompasses a therapeutically effective amount, which is an amount sufficient to provide a therapeutic benefit in the treatment of a disease, disorder or condition, or to delay or minimize one or more symptoms associated with the disease, disorder or condition. An effective amount also encompasses a prophylactically effective amount. In some embodiments, Compound 1 or a pharmaceutically acceptable salt thereof disclosed herein, or pharmaceutical compositions comprising the same is administered to the subject in an amount ranging from about 0.1 mg/kg to about 1 g/kg, such as from about 0.1 mg/kg to about 10 mg/kg, from about 0.1 mg/kg to about 5 mg/kg, from about 0.1 mg/kg to about 2.5 mg/kg, from about 0.1 mg/kg to about 1.5 mg/kg, from about 0.2 mg/kg to about 15 mg/kg, from about 0.2 mg/kg to about 5 mg/kg, from about 0.5 mg/kg to about 20 mg/kg, from about 0.5 mg/kg to about 10 mg/kg, or from about 0.5 mg/kg to about 5 mg/kg. In some embodiments, Compound 1 or a pharmaceutically acceptable salt thereof disclosed herein, or pharmaceutical compositions comprising the same is administered to the subject in an amount ranging from about 10 mg/kg to about 100 mg/kg, such as about 10, 15, 20, 25, 30, 35, 40, 45, 50, 55, 60, 65, 70, 75, 80, 85, 90, 95 or 100 mg/kg.

In some embodiments, Compound 1 or a pharmaceutically acceptable salt thereof disclosed herein, or pharmaceutical compositions comprising the same is administered to the subject as a single dose in an amount ranging from about 1 mg to about 180 mg or from about 2.5 mg to about 150 mg, such as about 1 mg, about 2 mg, about 3 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, or about 150 mg. In some embodiments, Compound 1 or a pharmaceutically acceptable salt thereof disclosed herein, or pharmaceutical compositions comprising the same is administered to the subject as a single dose in an amount of about 0.1 mg to about 500 mg (e.g., from about 0.5 mg to about 200 mg, from about 1 mg to about 150 mg, from about 5 mg to about 130 mg, or from about 10 mg to about 120 mg). In some embodiments, Compound 1 or a pharmaceutically acceptable salt thereof disclosed herein, or pharmaceutical compositions comprising the same is administered to the subject as a single dose in an amount of up to 150 mg, such as from about 30 mg to about 120 mg, such as about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, or about 120 mg. In some embodiments, Compound 1 or a pharmaceutically acceptable salt thereof disclosed herein, or pharmaceutical compositions comprising the same is administered to the subject as a single dose in an amount of about 90 mg or about 120 mg. In some embodiments, the dose is an oral dose.

In some embodiments, Compound 1 or a pharmaceutically acceptable salt thereof disclosed herein, or pharmaceutical compositions comprising the same is administered to the subject as multiple doses, with a maximum dose in an amount ranging from about 30 mg to about 150 mg, such as about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, or about 150 mg. In some embodiments, Compound 1 or a pharmaceutically acceptable salt thereof disclosed herein, or pharmaceutical compositions comprising the same is administered to the subject as multiple doses, with a maximum dose in an amount of about 90 mg or about 120 mg.

In some embodiments, Compound 1 or a pharmaceutically acceptable salt thereof disclosed herein, or pharmaceutical compositions comprising the same is administered to the subject orally. In some embodiments, Compound 1 or a pharmaceutically acceptable salt thereof disclosed herein, or pharmaceutical compositions comprising the same is administered to the subject every day. In some embodiments, Compound 1 or a pharmaceutically acceptable salt thereof disclosed herein, or pharmaceutical compositions comprising the same is administered to the subject every day for at least 14 days. In some embodiments, Compound 1 or a pharmaceutically acceptable salt thereof disclosed herein, or pharmaceutical compositions comprising the same is administered to the subject in ascending doses, with a starting dose of about 5 mg to about 150 mg, e.g., about 5 mg to about 25 mg, about 20 mg to about 100 mg, such as about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg or about 150 mg.

In some embodiments, Compound 1 or a pharmaceutically acceptable salt thereof disclosed herein, or pharmaceutical compositions comprising the same is administered to the subject in a fasted state, such as more than about 10 hours after the last meal and/or at least about 4 hours before the next meal. In some embodiments, Compound 1 or a pharmaceutically acceptable salt thereof disclosed herein, or pharmaceutical compositions comprising the same is administered to the subject in a fed state, such as after a meal normally consumed by the subject, including but is not limited to a high-fat and high calorie meal.

In some embodiments, Compound 1 or a pharmaceutically acceptable salt thereof disclosed herein, or pharmaceutical compositions comprising the same is orally administered to the subject in an amount effective to achieve a maximum plasma concentration (tₘₐₓ) between about 1.5 to about 5 hours, such as about 1.5 to about 4 hours, about 2.5 to about 5 hours, about 2.5 to about 4 hours, about 2 to about 4 hours, or about 2 to about 3 hours.

In some embodiments, Compound 1 or a pharmaceutically acceptable salt thereof disclosed herein, or pharmaceutical compositions comprising the same is orally administered to the subject in an amount effective to achieve plasma TC₅₀ (time to 50% of Compound 1 plasma concentration at plateau) of from about 1500 ng/g to about 800 ng/g, such as from about 1400 ng/g to about 900 ng/g, from about 1300 ng/g to about 1000 ng/g, from about 1200 ng/g to about 1100 ng/g, or from about 1150 ng/g to about 1100 ng/g. In some embodiments, Compound 1 or a pharmaceutically acceptable salt thereof disclosed herein, or pharmaceutical compositions comprising the same is orally administered to the subject in an amount effective to achieve plasma TC₅₀ of about 1500 ng/g, about 1400 ng/g, about 1350 ng/g, about 1300 ng/g, about 1250 ng/g, about 1200 ng/g, about 1150 ng/g, about 1100 ng/g, about 1050 ng/g, about 1000 ng/g, about 900 ng/g, about 950 ng/g, about 900 ng/g, about 850 ng/g, or about 800 ng/g.

In some embodiments, Compound 1 or a pharmaceutically acceptable salt thereof disclosed herein, or pharmaceutical compositions comprising the same is orally administered to the subject in an amount effective to achieve brain EC₅₀ (half maximal effective concentration of Compound 1) of from about 85 ng/g to about 50 ng/g, such as from about 85 ng/g to about 60 ng/g, from about 80 ng/g to about 65 ng/g, from about 75 ng/g to about 60 ng/g, from about 70 ng/g to about 60 ng/g, or from about 70 ng/g to about 65 ng/g. In some embodiments, Compound 1 or a pharmaceutically acceptable salt thereof disclosed herein, or pharmaceutical compositions comprising the same is orally administered to the subject in an amount effective to achieve brain EC₅₀ of about 85 ng/g, 80 ng/g, 75 ng/g, 70 ng/g, 65 ng/g, 60 ng/g, 55 ng/g, or 50 ng/g.

### Combination Therapy

Compound 1 or a pharmaceutically acceptable salt thereof disclosed herein, or pharmaceutical compositions comprising the same may be administered in combination with another agent or therapy. A subject to be administered a compound disclosed herein may have a disease, disorder, or condition, or a symptom thereof, that would benefit from treatment with another agent or therapy. These diseases or conditions can relate to epilepsy or an epilepsy syndrome.

In some embodiments, Compound 1 or a pharmaceutically acceptable salt thereof disclosed herein, or pharmaceutical compositions comprising the same is administered in combination with an anti-epilepsy agent. Anti-epilepsy agents include, but not limited to, brivaracetam, carbamazepine, clobazam, clonazepam, diazepam, divalproex, eslicarbazepine, ethosuximide, ezogabine, felbamate, gabapentin, lacosamide, lamotrigine, levetiracetam, lorazepam, oxcarbezepine, permpanel, phenobarbital, phenytoin, pregabalin, primidone, rufinamide, tigabine, topiramate, valproic acid, vigabatrin, zonisamide, and cannabidiol. In some embodiments, Compound 1 or a pharmaceutically acceptable salt thereof disclosed herein, or pharmaceutical compositions comprising the same is administered in combination with carbamazepine.

In some embodiments, the disclosed methods comprise administering to the subject in need thereof Compound 1 or a pharmaceutically acceptable salt thereof disclosed herein, or pharmaceutical compositions comprising the same in combination with an anti-epilepsy agent. In some embodiments, the disclosed methods comprise administering to the subject in need thereof Compound 1 or a pharmaceutically acceptable salt thereof disclosed herein, or pharmaceutical compositions comprising the same in combination with carbamazepine.

Accordingly, one aspect of the disclosure provides for a composition comprising Compound 1 or a pharmaceutically acceptable salt thereof disclosed herein and at least one additional therapeutic agent. In some embodiments, the composition comprises Compound 1 or a pharmaceutically acceptable salt thereof disclosed herein and at least two additional therapeutic agents. In some embodiments, the composition comprises Compound 1 or a pharmaceutically acceptable salt thereof disclosed herein and at least three additional therapeutic agents, Compound 1 or a pharmaceutically acceptable salt thereof disclosed herein and at least four additional therapeutic agents, or Compound 1 or a pharmaceutically acceptable salt thereof disclosed herein and at least five additional therapeutic agents.

The methods of combination therapy include co-administration of a single formulation containing Compound 1 or a pharmaceutically acceptable salt thereof disclosed herein and additional therapeutic agent or agents, essentially contemporaneous administration of more than one formulation comprising Compound 1 or a pharmaceutically acceptable salt thereof disclosed herein and additional therapeutic agent or agents, and consecutive administration of Compound 1 or a pharmaceutically acceptable salt thereof disclosed herein and therapeutic agent or agents, in any order, wherein preferably there is a time period where Compound 1 or a pharmaceutically acceptable salt thereof disclosed herein and additional therapeutic agent or agents simultaneously exert a therapeutic effect.

### Dosage Forms and Compositions

In one aspect, the disclosure provides dosage forms or compositions useful for treating a disease, disorder, or condition described herein, e.g., a neurological disorder, a disorder associated with excessive neuronal excitability, or a disorder associated with de novo gain-of-function or loss-of-function mutations in major central nervous system sodium channel genes, such as for example, SCN1A, SCN2A, and SCN8A.

Accordingly, the disclosure provides pharmaceutical compositions that contain, as the active ingredient, Compound 1 or a pharmaceutically acceptable salt thereof disclosed herein and one or more pharmaceutically acceptable excipients, carriers, including inert solid diluents and fillers, diluents, including sterile aqueous solution and various organic solvents, permeation enhancers, solubilizers and adjuvants. The pharmaceutical compositions may be administered alone or in combination with other therapeutic agents. Such compositions are prepared in a manner well known in the pharmaceutical art (see, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

The pharmaceutical compositions may be administered in either single or multiple doses by any of the accepted modes of administration of agents having similar utilities, for example as described in those patents and patent applications incorporated by reference, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer. In some embodiments, the compounds or pharmaceutical compositions of the disclosure are administered orally.

One mode for administration is parenteral, particularly by injection. The forms in which the compositions of the disclosure may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection, but less preferred in the context of the present invention. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating a compound according to the present invention in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Oral administration is another route for administration of compounds in accordance with the present disclosure. Administration may be via capsule or tablets, or the like. In making the pharmaceutical compositions that include at least one compound described herein, the active ingredient is usually diluted by an excipient and/or enclosed within such a carrier that can be in the form of a capsule, sachet, paper or other container. When the excipient serves as a diluent, it can be in the form of a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include lubricating agents such as talc, magnesium stearate, and mineral oil; wetting agents; emulsifying and suspending agents; preserving agents such as methyl and propylhydroxy-benzoates; sweetening agents; and flavoring agents.

The compositions of the disclosure can be formulated so as to provide quick, sustained or delayed release of the active ingredient after administration to the patient by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

The compositions are preferably formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient (e.g., a tablet, capsule, ampoule). The compounds are generally administered in a pharmaceutically effective amount. Preferably, for oral administration, each dosage unit contains from about 1 mg to about 2 g of a compound described herein, and for parenteral administration, preferably from about 0.1 to about 700 mg of a compound described herein. It will be understood, however, that the amount of the compound actually administered usually will be determined by a physician, in the light of the relevant circumstances, including the condition to be treated, the chosen route of administration, the actual compound administered and its relative activity, the age, weight, and response of the individual subject, the severity of the subject's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules.

The tablets or pills of the present disclosure may be coated or otherwise compounded to provide a dosage form affording the advantage of prolonged action, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably, the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

In one aspect, provided herein is a dosage form or a composition in a dosage form comprising: from about 0.1 mg to about 500 mg (e.g., from about 0.5 mg to about 200 mg, from about 1 mg to about 150 mg, from about 10 mg to about 120 mg) of Compound 1 or a pharmaceutically acceptable salt thereof disclosed herein and a pharmaceutically acceptable excipient.

In some embodiments, the dosage form or a composition in a dosage form comprises from about 2.5 mg to about 150 mg (e.g., from about 10 mg to about 150 mg, from about 20 mg to about 150 mg, from about 40 mg to about 150 mg, from about 60 mg to about 150 mg, from about 80 mg to about 150 mg, from about 100 mg to about 150 mg, from about 10 mg to about 120 mg, from about 20 mg to about 120 mg, from about 40 mg to about 120 mg, from about 60 mg to about 120 mg, from about 80 mg to about 120 mg, from about 100 mg to about 120 mg, from about 10 mg to about 100 mg, from about 20 mg to about 100 mg, from about 40 mg to about 100 mg, from about 60 mg to about 100 mg, from about 80 mg to about 100 mg, from about 10 mg to about 80 mg, from about 20 mg to about 80 mg, from about 40 mg to about 80 mg, from about 60 mg to about 80 mg, from about 10 mg to about 60 mg, from about 20 mg to about 60 mg, from about 40 mg to about 60 mg, from about 70 mg to about 120 mg, from about 70 mg to about 100 mg, from about 50 mg to about 120 mg, from about 50 mg to 90 mg, from about 30 mg to about 120 mg, from about 30 mg to about 60 mg, from about 30 mg to about 80 mg, from about 30 mg to about 100 mg) of Compound 1 or a pharmaceutically acceptable salt thereof disclosed herein. In some embodiments, the dosage form is an oral dosage form.

In some embodiments, the dosage form or a composition in a dosage form comprises from about 1 mg to about 100 mg (e.g., 1 from about 1 mg to about 80 mg, from about 1 mg to about 50 mg, from about 1 mg to about 20 mg, from about 1 mg to about 10 mg, from about 1 mg to about mg, from about 5 mg to about 100 mg, from about 5 mg to about 80 mg, from about 5 mg to about 50 mg, from about 5 mg to about 20 mg) of Compound 1 or a pharmaceutically acceptable salt thereof disclosed herein.

In some embodiments, the dosage form or a composition in a dosage form comprises about 200 mg, 190 mg, 180 mg, 170 mg, 160 mg, 150 mg, 140 mg, 130 mg, 120 mg, 110 mg, 100 mg, about 99 mg, about 98 mg, about 97 mg, about 96 mg, about 95 mg, about 94 mg, about 93 mg, about 92 mg, about 91 mg, about 90 mg, about 85 mg, about 80 mg, about 75 mg, about 70 mg, about 69 mg, about 68 mg, about 67 mg, about 66 mg, about 65 mg, about 64 mg, about 63 mg, about 62 mg, about 61 mg, about 60 mg, about 59 mg, about 58 mg, about 57 mg, about 56 mg, about 55 mg, about 54 mg, about 53 mg, about 52 mg, about 51 mg, about 50 mg, about 45 mg, about 40 mg, about 35 mg, about 30 mg, about 25 mg, about 20 mg, about 15 mg, about 10 mg, about 7 mg, about 5 mg, about 2.5 mg, about 2 mg, about 1.5 mg, or about 1 mg of Compound 1 or a pharmaceutically acceptable salt thereof disclosed herein. In some embodiments, the dosage form or a composition in a dosage form is for oral administration.

In another aspect, the present disclosure provides a dosage form or a composition in a dosage form comprising: a plurality of particles of Compound 1 or a pharmaceutically acceptable salt thereof disclosed herein and a pharmaceutically acceptable excipient, wherein the amount of the plurality of particles of Compound 1 or a pharmaceutically acceptable salt thereof disclosed herein in the dosage form is from about 0.1 mg to about 500 mg (e.g., from about 0.5 mg to about 200 mg, from about 1 mg to about 150 mg, from about 10 mg to about 120 mg).

In some embodiments, the plurality of particles of Compound 1 or a pharmaceutically acceptable salt thereof disclosed herein in the dosage form or composition is from about 2.5 mg to about 150 mg (e.g., from about 10 mg to about 150 mg, from about 20 mg to about 150 mg, from about 70 mg to about 120 mg, from about 30 mg to about 60 mg, about 100 mg, about 50 mg).

In some embodiments, the dosage form or the composition is configured for oral administration. In some embodiments, the dosage form is a solid form. In some embodiments, the dosage form is in the form of a capsule. In some embodiments, the pharmaceutical excipient in the capsule is a filler (e.g., cellulose derivatives (e.g., microcrystalline cellulose), starches (e.g., hydrolyzed starches, and partially pregelatinized starches), anhydrous lactose, lactose monohydrate, sugar alcohols (e.g., sorbitol, xylitol, and mannitol).

In some embodiments, the dosage form is a liquid form. In some embodiments, the dosage form is in the form of a solution. In some embodiments, the pharmaceutical excipient in the solution is selected from the group consisting of a filler (e.g., polymer (e.g., PEG 400)), an emulsifier (e.g., a castor oil derivative (e.g., Kolliphor RH40), a surfactant (e.g., a glyceride (e.g., Labrafil M2125 CS), a vitamin derivative (e.g., Vitamin ETPGS)), a solvent (e.g., propylene glycol, ethanol, diethylene glycol monoethyl ether (or Transcutol HP)).

In some embodiments, the concentration of Compound 1 or a pharmaceutically acceptable salt thereof disclosed herein in the solution is from about 0.1 mg/mL to about 10 mg/mL (e.g., from about 0.5 mg/mL to about 10 mg/mL, from about 1 mg/mL to about 10 mg/mL, from about 2 mg/mL to about 10 mg/mL, from about 3 mg/mL to about 10 mg/mL, from about 4 mg/mL to about 10 mg/mL, from about 5 mg/mL to about 10 mg/mL, from about 6 mg/mL to about 10 mg/mL, from about 0.1 mg/mL to about 8 mg/mL, from about 0.5 mg/mL to about 8 mg/mL, from about 1 mg/mL to about 8 mg/mL, from about 2 mg/mL to about 8 mg/mL, from about 3 mg/mL to about 8 mg/mL, from about 4 mg/mL to about 8 mg/mL, from about 5 mg/mL to about 8 mg/mL, from about 6 mg/mL to about 8 mg/mL, from about 0.5 mg/mL to about 6 mg/mL, from about 1 mg/mL to about 6 mg/mL, from about 2 mg/mL to about 6 mg/mL, from about 3 mg/mL to about 6 mg/mL, from about 4 mg/mL to about 6 mg/mL, from about 0.5 mg/mL to about 4 mg/mL, from about 1 mg/mL to about 4 mg/mL, or from about 2 mg/mL to about 4 mg/mL).

In some embodiments, the concentration of Compound 1 or a pharmaceutically acceptable salt thereof disclosed herein in the solution is about 0.1 mg/mL, about 0.5 mg/mL, about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, or about 10 mg/mL.

In some embodiments, the dosage form is in the form of a suspension. In some embodiments, the concentration of Compound 1 or a pharmaceutically acceptable salt thereof disclosed herein in the suspension is about 0.1 mg/mL, about 0.5 mg/mL, about 1 mg/mL, about 1.5 mg/mL, about 2 mg/mL, about 2.5 mg/mL, about 3 mg/mL, about 3.5 mg/mL, about 4 mg/mL, about 4.5 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 20 mg/mL, about 25 mg/mL.

In some embodiments, the concentration of Compound 1 or a pharmaceutically acceptable salt thereof disclosed herein in the suspension is from about 0.1 mg/mL to about 10 mg/mL (e.g., from about 0.5 mg/mL to about 10 mg/mL, from about 1 mg/mL to about 10 mg/mL, from about 2 mg/mL to about 10 mg/mL, from about 3 mg/mL to about 10 mg/mL, from about 4 mg/mL to about 10 mg/mL, from about 5 mg/mL to about 10 mg/mL, from about 6 mg/mL to about 10 mg/mL, from about 0.1 mg/mL to about 8 mg/mL, from about 0.5 mg/mL to about 8 mg/mL, from about 1 mg/mL to about 8 mg/mL, from about 2 mg/mL to about 8 mg/mL, from about 3 mg/mL to about 8 mg/mL, from about 4 mg/mL to about 8 mg/mL, from about 5 mg/mL to about 8 mg/mL, from about 6 mg/mL to about 8 mg/mL, from about 0.5 mg/mL to about 6 mg/mL, from about 1 mg/mL to about 6 mg/mL, from about 2 mg/mL to about 6 mg/mL, from about 3 mg/mL to about 6 mg/mL, from about 4 mg/mL to about 6 mg/mL, from about 0.5 mg/mL to about 4 mg/mL, from about 1 mg/mL to about 4 mg/mL, or from about 2 mg/mL to about 4 mg/mL).

### EXAMPLES

In order that the embodiments described herein may be more fully understood, the following examples are set forth. The examples described in this application are offered to illustrate the compounds, pharmaceutical compositions, and methods provided herein and are not to be construed in any way as limiting their scope.

### Example 1. Effect of Compound 1 on Mouse Wildtype CA1 Pyramidal Neuron Excitability Using Brain Slice Whole-Cell Patch-Clamp Electrophysiology

The aim of this study was to determine the effect of Compound 1 on intrinsic excitability of CA1 pyramidal neurons from brain slices obtained from wildtype mice.

Mice (p17-p21) were anesthetized with 2% isoflurane. The brain was removed and placed into an iced slurry of brain slice cutting solution containing: 125 mM Choline chloride, 2.5 mM KCl, 1.25 mM NaH₂PO₄, 26 mM NaHCO₃, 20 mM D-glucose, 0.4 mM CaCl₂·2H₂O, and 6 mM MgCl₂·6H₂O at a pH of 7.4 maintained by continuous bubbling with carbogen gas (95% 02-5% CO₂). Three hundred micrometer coronal hippocampal slices were cut on a vibratome (VT1200; Leica) for whole-cell patch-clamp experiments. Slices were incubated for a minimum of 1 hour in the brain slice cutting solution at room temperature before patching.

Brain slices were transferred to a submerged recording chamber on an upright microscope (Slicescope Pro 1000; Scientifica) and were perfused (2 ml/min) with the extracellular artificial cerebral spinal fluid (aCSF) recording solution at 32° C. The extracellular aCSF recording solution contained: 125 mM NaCl, 2.5 mM KCl, 1.25 mM NaH₂PO₄, 26 mM NaHCO₃, 10 mM D-glucose, 2 mM CaCl₂·2H₂O, and 2 mM MgCl₂·6H₂O at a pH of 7.4 maintained by continuous bubbling with carbogen gas (95% 02-5% CO₂).

CA1 pyramidal neurons were identified visually in the stratum pyramidale of the CA1 region of the hippocampus, using infrared-oblique illumination microscopy with a 40× water-immersion objective (Olympus) using a camera (Dage IR-2000; Dage). Cell identity was also confirmed using action potential firing characteristics, where action potentials were accommodating at high current injections and had a wide action potential half-width. Patch-clamp recordings were made using a micromanipulator (MPC-200; Sutter) and Axon Multiclamp 700B patch-clamp amplifier (MDS). Data were acquired using pClamp software (v10; MDS) using a sampling rate of 50 kHz and low pass Bessel filtered at 10 kHz (Digidata 1550b; Axon). Patch pipettes (3-7 MΩ; GC150F-7.5; Harvard Instruments) pulled using a Flaming/brown micropipette puller (Model P-1000; Sutter) were filled with intracellular recording solution containing: 125 mM K gluconate, 5 mM KCl, 2 mM MgCl₂·6H₂O, 10 mM HEPES, 4 mM ATP-Mg, 0.3 mM GTP-Na, 10 mM Phosphocreatine, 0.1 mM EGTA, and 0.2% Biocytin at a pH of 7.2 (adjusted with KOH) and a measured osmolarity of 292 mOsm.

Studies were performed using whole-cell current clamp recording mode. Once whole-cell configuration was obtained for 2 minutes, a holding current was injected to maintain a membrane potential of approximately −70 mV. Current steps (injected current of between −60 to 340 pA in 20 pA steps, 400 ms duration) were applied in current clamp mode. The amplitude of current injections was relative to the holding current. A test pulse (−5 pA amplitude, 50 ms duration) was applied 650 ms after the termination of the main current step. The inter sweep interval was 5 seconds (0.2 Hz). To be included in the study, a cell had to have an access resistance of less than 20 MΩ and a holding current of less than −200 pA. Once baseline action potential firing was determined in the presence of the extracellular aCSF recording solution (baseline), Compound 1 (300 nM or 3 μM) was washed onto the slice for 5 minutes before repeating the action potential generating protocol.

Data were analyzed using Axograph X software. Individual action potentials were identified and counted using a +50 mV amplitude threshold relative to pre-event baseline. The frequencies of action potentials generated were plotted for each of the current injections to create an input-frequency relationship for each cell. Once an action potential count had less than three cells contributing to the averaged amplitude, it was excluded from analysis. Statistical analysis was performed using GraphPad Prism software (v8). Paired two-tailed Student's t-tests were used to test the effect of compound on action potential firing compared to baseline. In all cases the significance for analysis was set as an alpha value of 0.05.

### Example 2. Effect of Compound 1 on Human Na_{V}1.6 Channel Using the PatchXpress® (Molecular Devices) Electrophysiology Platform

The aim of this study was to determine the effect of Compound 1 on the human Na_{V}1.6 late (persistent) current and peak current tonic block (TB), and peak current use-dependent block (UDB).

### 1. Materials and Methods

i. Cell Preparation

HEK-293 cell lines stably expressing human Na_{V}1.6 (NP_055006) were used. All cells were seeded at 2×10⁶ cells per Nunc T75 flask for 2 days in culture. At time of harvest for assays the cell counts were approximately 6×10⁶ cells. Cells were washed (1×) in DPBS (Hyclone, Cat #SH30028.03) for approximately 30 seconds. 1 mL of 1×0.05% Trypsin-EDTA (GIBCO Cat #25300-054) was added and swirled around to cover the bottom of the flask and was allowed to sit on the cells for approximately 4 minutes (±90% of the cells were lifted by light tapping of the flask). 10 mL of warmed media (DMEM high glucose media Hyclone, SH30022.02 supplemented with 10% Fetal Bovine Serum, 2 mM Sodium Pyruvate, 10 mM HEPES and 400 μg/mL G418) was added to inactivate the trypsin. Cells were triturated until a single cell suspension was achieved. A cell count was performed, and cells were aliquoted into a 250 ml centrifuge tube at a concentration of 2×10⁵/mL in ±30 ml of prewarmed media. The 250 mL centrifuge tube was placed on a rocker in an incubator set at 28° C. and gently rocked for approximately 1 hour to allow cells to recover. Cell aliquots of 5 mL (1×10⁶ cells) were placed in a 15 mL centrifuge tube and spun at 100×g for 2 minutes. Supernatant was removed leaving a cell pellet. 100 μL of external recording solution was added to the pellet and triturated 20 times to achieve a single cell suspension and then transferred to a 1.5 mL tube for placement in the PatchXpress® (Molecular Devices).

ii. Test Agent Preparation

Compound 1 was provided as powder and prepared as 10 mM DMSO stocks in 1 dram glass vials prior to assay. Immediately prior to assay, Compound 1 was diluted with DMSO to 300× the designated final assay concentration. Assay dilutions (1× concentration) were prepared by pipetting 3 μL of diluted Compound 1 into 897 μL of extracellular solution in a 1 mL glass shell vial. Vials were capped and vortexed until initiation of the PatchXpress® (Molecular Devices) recording protocol.

iii. PatchXpress® (Molecular Devices) Recording Solutions

The same intracellular recording solution was used for the persistent and peak I_{Na} assays, which contained: 135 mM CsF, 10 mM CsCl, 5 mM NaCl, 10 mM HEPES, 5 mM EGTA at a pH of 7.4 (adjusted with CsOH) and a measured osmolarity of 298 mOsm (adjusted with mannitol). For the persistent I_{Na} assay, the external recording solution contained: 135 mM NaCl, 5.4 mM KCl, 5 mM glucose, 2 mM CaCl₂), 1 mM MgCl₂, 10 mM HEPES, and 200 nM ATX-II (sea anemone toxin, Alomone Labs; Jerusalem, Israel) at a pH of 7.4 (adjusted with NaOH) and a measured osmolarity of 300 mOsm (adjusted with mannitol). For the peak I_{Na} assays, the external recording solution contained: 100 mM NaCl, 35 mM NMDG, 5.4 mM KCl, 5 mM glucose, 2 mM CaCl₂), 1 mM MgCl₂, and 10 mM HEPES at a pH of 7.4 (adjusted with NaOH) and a measured osmolarity of 300 mOsm (adjusted with mannitol).

iv. Experimental Protocols

All studies were performed using the PatchXpress® (Molecular Devices) automated patch clamp platform (Molecular Devices) using the whole cell configuration. Recordings were performed at room temperature. Data was collected using Patch Commander software (Molecular Devices) and processed using DataXpress 2.0 (Molecular Devices). Cell acceptance criteria were continuously monitored by custom scripts during the recording. Acceptance criteria for all assays was: baseline peak I_{Na}>800 pA, Rₛₑₐₗ>200 MΩ and, where appropriate, persistent I_{Na}>100 pA. Compensation was 50% and leak subtraction was not used. Currents were filtered at 5 kHz and digitized at 32.5 kHz. R_{access} and Rₛₑₐₗ were monitored throughout the recording and cells with R_{access}>10 MΩ or Rₛₑₐₗ<200 MΩ were terminated automatically by script.

Late (persistent) current Block: The inhibition of ATX-II activated late current was measured using a 200 ms voltage step to 0 mV. Protocols used low stimulation rates (about 0.1 Hz) and negative potentials (−120 mV) to keep channels in closed (resting) states. Pharmacology was measured as the mean I_{Na} during the final 20 ms of the step to 0 mV and leak subtraction was not used.

Tonic Block (TB): Tonic Block (TB) protocols used low stimulation rates (about 0.1 Hz) and negative potentials (−120 mV) to keep channels in closed (resting) states. Pharmacology measured during peak I_{Na} in response to step to 0 mV. No leak subtraction was used.

Use Dependent Block (UDB-10 Hz): UDB protocols used elevated stimulation rates (10 Hz) to cycle channels between closed (resting), open, and fast inactivated states. Slow inactivation was minimized by using a negative potential (−120 mV) between the steps. Pharmacology measured during peak I_{Na} in response to step to 0 mV. No leak subtraction was used.

Plotting and fitting were performed using GraphPad Prism (GraphPad Software). Percent inhibition was calculated, expressed as mean±SEM, and plotted versus the tested concentration. Data were fit using a Hill equation [Max_Effect/(1+(IC₅₀/x)\hat{ }Hill_Slope] to estimate the concentration of compound producing half inhibition (IC₅₀) and hill slope. MaxEffect was allowed to vary for the Use Dependent Block (UDB) assay but was fixed at 100 for all other assays.

### 2. Results

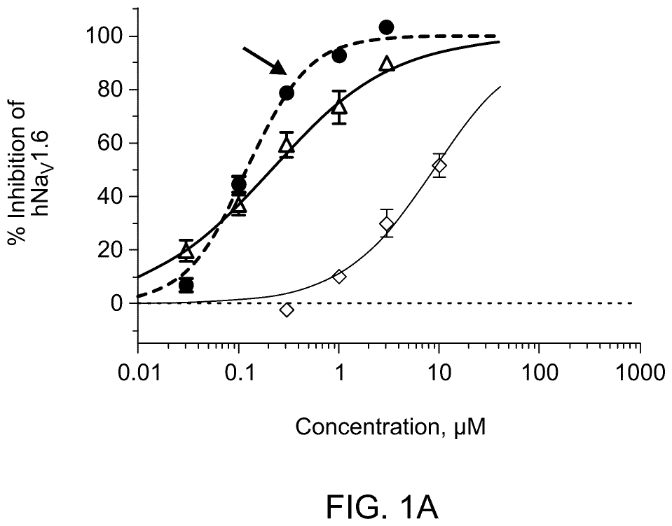

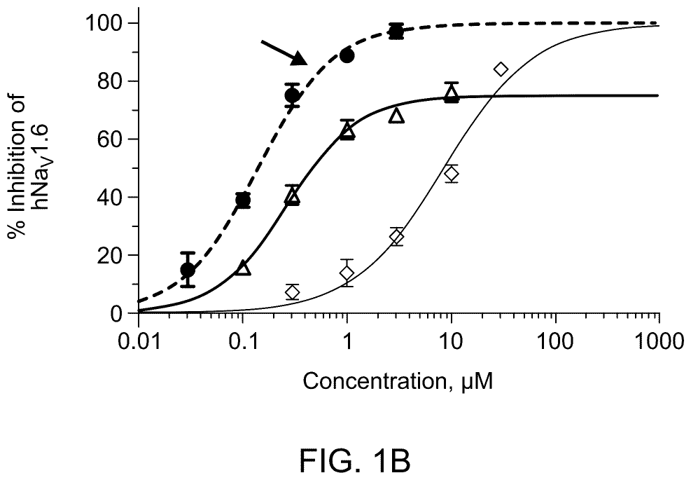

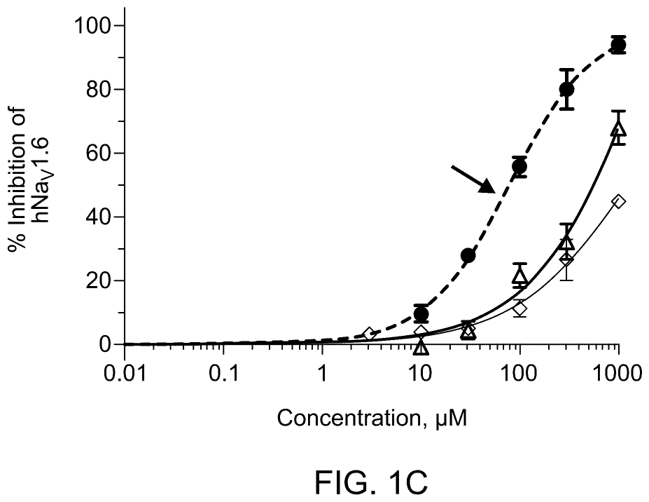

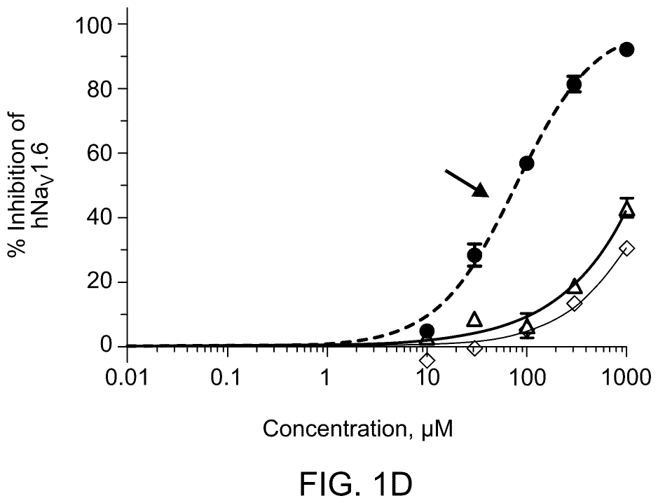

Compound 1 was found to produce a concentration dependent inhibition of late sodium current induced by the application of ATX-II in human Na_{V}1.6 channel (FIG. 1A). When compared to Reference Compound that is also under investigation as a potential sodium channel blocker, Compound 1 appeared to have a different “Na_{V} Fingerprint” (compare FIG. 1A (Compound 1) and FIG. 1B (Reference Compound)). Nonetheless, similar to Reference Compound, Compound 1 demonstrated preference for persistent I_{Na} relative to peak I_{Na} for all assay conditions (arrow in FIG. 1A). By contrast, the two standard Na_{V}-targeting antiepileptic drugs lamotrigine (LTG) and carbamazepine (CBZ) exhibited lower potency and no preference for persistent I_{Na} (arrows in FIG. 1C (LTG) and FIG. 1D (CBZ)).

### Example 3. Effect of Compound 1 on Maximal Electroshock Induced Seizure in Mouse Model

The maximal electroshock (MES) test is a validated model for evaluating anti-seizure potential of compounds. Valproic acid (VPA) is used clinically for its anticonvulsant properties and is an effective treatment for tonic seizures. The object of these studies was to evaluate the effect of Compound 1 (0.3, 1, 3 and 10 mg/kg p.o. (oral)) to attenuate MES-induced seizures in male CD-1 mice.

### 1. Materials and Methods

i. Animals

Male CD-1 mice, 6-weeks old, were obtained from Vital River (Beijing, China). The average body weight was approximately 35 g at the time the experiments were carried out. The mice were housed in groups of 3-5 under controlled conditions (temperature: 20-26° C.; humidity: 40-70%; air exchange rate: 10-15 cycles/hour; 12:12 light-dark cycle with lights on at 5:00 a.m.). Food and water were available ad libitum. The mice were acclimated to these conditions for 6 days before the start of behavioral testing.

ii. Drug Formulation

Dosing solutions were prepared on each experiment day. A stock solution of the highest dose of Compound 1 was prepared in 35% HPBCD (Vehicle 2) and diluted to make the lower doses. VPA was prepared in saline (Vehicle 1). All solutions were protected from light. All compounds and vehicles were dosed at 10 ml/kg.

iii. Study Protocol

The MES test was carried out over two days with half of the mice from each treatment group being tested on each day. Mice were brought to the test room at least one hour before the start of the experiment. All animals were marked on the tail with a permanent marker and weighed. Mice were randomly assigned into one of the six treatment groups.

Animals were p.o. (oral) administered Compound 1 or 35% HPBCD (Vehicle 2) 30 minutes prior to the MES test or i.p. administered VPA or saline (Vehicle 1) 30 minutes prior to the MES test. Just before the start of the MES test, possible side effects such as overt sedation were recorded.

A Grass S88x (Grass Technologies of Astro-Med, Inc., West Warwick, RI) Stimulus Isolation Unit (A385, WPI Inc., USA) was set to deliver a 50 mA square-wave stimulus, with a 0.8 second duration, a pulse width of 10 msec, and a frequency of 50 Hz. A pair of custom stainless steel electrodes were soaked in 0.2% Agar, and then the subject received bilateral transauricular stimulation through ear-clip electrodes. During the stimulation, mice were manually restrained. They were then released into the observation cage for convulsion observation for 60 seconds immediately after stimulation. Each mouse was observed continuously by individuals blinded to treatment conditions and results were recorded.

Sedation assessment endpoints were: (1) None: mouse exhibits normal locomotor behavior; (2) Mild: mouse shows less locomotion or immobility when alone in its home cage, but shows normal locomotor activity if provoked by touching from an observer's hand; (3) Moderate: mouse shows immobility when alone in its home cage and reduced locomotor activity when pushed or provoked by touching from an observer's hand; and (4) Severe: mouse completely loses the ability to move.

Anti-seizure assessment endpoints were: (1) latency of hindlimb tonic flexion; (2) latency of hindlimb tonic extension; (3) total #hindlimb tonic flexions; (4) total #hindlimb tonic extensions; (5) latency to death; and (6) mortality rate.

Following the experiment, mice were anesthetized with CO₂ and terminal plasma and brain tissue samples were collected. 500 μL whole blood was collected via cardiac puncture and placed in tubes with 10 μL EDTAK2. The tubes were then placed in wet ice until centrifuged at 2,000 g for 5 minutes at 4° C. The supernatant plasma was pipetted into Eppendorf tubes. Both brain and plasma samples were stored at −80° C. until determination of Compound 1 concentration in each sample.

iv. Plasma Sample Preparation

An aliquot of 20 μL unknown sample, calibration standard, quality control, dilute quality control, single blank and double blank sample was added to a 1.5 mL tube. Each sample (except the double blank) was quenched with 300 μL IS solution respectively (double blank sample was quenched with 300 μL ACN), and then the mixture was vortex-mixed well (at least 15 seconds) and centrifuged for 15 minutes at 12000 g, 4° C. 70 μL supernatant was transferred to a 96-well plate and centrifuged for 5 minutes at 3220 g, 4° C. Then 5 μL supernatant was injected for LC-MS/MS analysis.

v. Brain Sample Preparation

Brain homogenate was prepared by homogenizing brain tissue with 5 volumes (w:v) of homogenizing solution (cold 15 mM PBS/MeOH (V:V, 2:1)). An aliquot of 20 μL unknown sample, calibration standard, quality control, dilute quality control, single blank and double blank sample was added to a 1.5 mL tube. Each sample (except the double blank) was quenched with 300 μL IS solution respectively (double blank sample was quenched with 300 μL ACN), and then the mixture was vortex-mixed well (at least 15 seconds) and centrifuged for 15 minutes at 12000 g, 4° C. 70 μL supernatant was transferred to the 96-well plate and centrifuged for 5 minutes at 3220 g, 4° C. Then 5 μL supernatant was injected for LC-MS/MS analysis.

vi. Statistical Analysis

All statistical analyses were conducted using GraphPad Prism 7.0. Data are presented as mean±s.e.m. and p<0.05 was regarded as statistically significant. Mann-Whitney test was used to detect significant differences in latency and number of seizures between saline and VPA groups. Kruskal-Wallis followed by Dunn's test was used to detect significant differences in latency and number of seizures between 35% HPBCD and Compound 1.

vii. Calculating ED₅₀ and EC₅₀ Values

Dose-response and concentration-response curves for plasma and brain were fitted for each endpoint using GraphPad Prism. From these fitted curves, ED₅₀ and EC₅₀ values were calculated.

### 2. Results

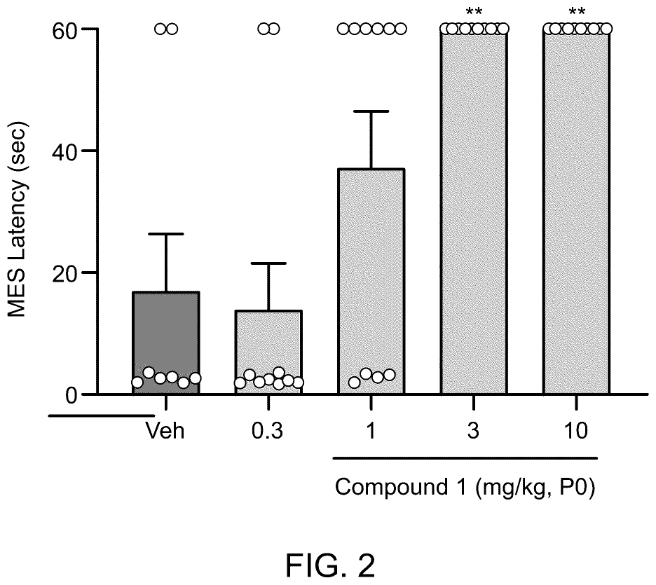

As shown in FIG. 2, at 1 mg/kg (p.o.), Compound 1 significantly increased latency to seizures following bilateral transauricular stimulation. The ability of Compound 1 to increase latency to seizures following MES was even more pronounced at 3 and 10 mg/kg (p.o.). Compound 1 has a calculated ED₅₀ of 0.67 mg/kg. These data suggest that Compound 1 has anti-seizure properties in the MES-induced seizure model.

### Example 4. Effect of Compound 1 on Spontaneous Locomotor Activity (sLMA) in Mouse Model

The Spontaneous Locomotor Activity (sLMA) test is a validated model for evaluating the potential motor side effects of compounds. The objective of this study was to evaluate the effect of Compound 1 (3, 5.6, 10 and 20 40 mg/kg, p.o.) on spontaneous locomotor activity (sLMA) in male CD-1 mice at 30 minutes post-dose.

### 1. Materials and Methods

i. Animals

Male CD-1 mice, 6-weeks old, were obtained from Vital River (Beijing, China). The average body weight was 25-35 g at the time the experiments were carried out. The mice were housed in groups of 3-5 under controlled conditions (temperature: 20-26° C.; humidity: 40-70%; air exchange rate: 10-15 cycles/hour; 12:12 light-dark cycle with lights on at 5:00 a.m.). Food and water were available ad libitum. The mice were acclimated to these conditions for 6 days before the start of the studies.

ii. Drug Formulation

Dosing solutions were prepared on each experiment day. A stock solution of the highest dose of Compound 1 was prepared in 35% HPBCD (vehicle) and diluted to make the lower doses. The stock was stirred and sonicated for at least 20 minutes and resulted in a homogeneous suspension. All solutions were protected from light. Samples from dosing solutions were stored at 4° C. Both compound and vehicle were dosed at 10 ml/kg.

iii. Study Protocol

The sLMA test was carried out over two days. Half of the mice from each treatment group was tested on each day. All animals were marked on the tail with a permanent marker and weighed. Mice were randomly assigned into one of the four treatment groups. Animals were acclimated to the test room at least 30 minutes before the start of the experiment.

Animals were p.o. administered Compound 1 or 35% HPBCD (vehicle) 30 minutes prior to the sLMA test. Possible side effects such as overt sedation were recorded just before the start of the sLMA test.

Sedation assessment endpoints were: (1) None: mouse exhibits normal locomotor behavior; (2) Mild: mouse shows less locomotion or immobility when alone in its home cage, but shows normal locomotor activity if provoked by touching from an observer's hand; (3) Moderate: mouse shows immobility when alone in its home cage and reduced locomotor activity when pushed or provoked by touching from an observer's hand; and (4) Severe: mouse completely loses the ability to move.

30 minutes after being dosed with Compound 1 or vehicle, the mouse was placed at the center of the test chamber (40×40×30 cm, 45±5 Lux on the floor) for the 30-minute sLMA video recording. Each mouse was automatically tracked with overhead cameras using a 1-minute sampling window in an isolated chamber. Spontaneous locomotor activity was then analyzed offline using the Animal Behavior Video Tracking Analysis System (Ji Liang Software Technology Co., Ltd., Shanghai, China).

Locomotor status was defined as >2 mm of movement in every 200 ms (Frame-rate recorded was 20 frames/second. Locomotor status was identified in every 4 frame-to-frame interval. The accumulated shift of tracking spots that exceeded 2 mm in every 4 frame intervals was identified as a locomotor epoch). Traveling distance was calculated automatically from all the locomotion epochs and analyzed. Following the test, a short video recording (10 seconds) was taken of 2-3 representative mice showing side effects from each of the Compound 1 treatment groups.

All mice were anesthetized with CO₂. Then, terminal plasma and brain tissue samples were collected from animals in the drug treatment groups. 500 μL whole blood was collected via cardiac puncture and placed in tubes with 10 μL EDTAK2. The tubes were then placed in wet ice until centrifuged at 2000 g for 5 minutes at 4° C. The supernatant plasma was pipetted into Eppendorf tubes. Both brain and plasma samples were stored at −80° C. until drug concentration analysis of Compound 1 levels.

iv. Plasma Sample Preparation

An aliquot of 20 μL unknown sample, calibration standard, quality control, dilute quality control, single blank and double blank sample was added to a 1.5 mL tube. Each sample (except the double blank) was quenched with 300 μL IS solution (double blank sample was quenched with 300 μL ACN), and then the mixture was vortex-mixed well for at least 15 seconds and centrifuged for 15 minutes at 12000 g, 4° C. 65 μL supernatant was transferred to a 96-well plate and centrifuged for 5 minutes at 3220 g, 4° C. Then 3 μL supernatant was directly injected for LC-MS/MS analysis.

v. Brain Sample Preparation

Brain homogenate was prepared by homogenizing brain tissue with 5 volumes (w:v) of cold 15 mM PBS/MeOH (V:V, 2:1). An aliquot of 40 μL unknown sample, calibration standard, quality control, dilute quality control, single blank and double blank sample was added to a 1.5 mL tube. Each sample (except the double blank) was quenched with 600 μL IS solution (double blank sample was quenched with 600 μL ACN), and then the mixture was vortex-mixed well for at least 15 seconds and centrifuged for 15 minutes at 12000 g, 4° C. 65 μL supernatant was transferred to a 96-well plate and centrifuged for 5 minutes at 3220 g, 4° C. Then 3 μL of the supernatant was directly injected for LC-MS/MS analysis.

vi. Statistical Analysis

All statistical analyses were conducted using GraphPad Prism 7.0. Data are presented as mean±s.e.m., and p<0.05 was regarded as statistically significant. Two-way ANOVA with Dunnett's post hoc test was used to detect significant differences in traveling distance per 5-minute bins between vehicle and Compound 1 treatment groups. ANOVA followed by Dunnett's test was used to detect significant differences in total traveling distance over 30 minutes between vehicle and Compound 1 treatment groups.

vii. Calculating TD₅₀ and TC₅₀ Values

Dose-response and concentration-response curves for plasma and brain were fitted for each endpoint using GraphPad Prism. From these fitted curves, TD₅₀ and TC₅₀ values were calculated.

### 2. Results

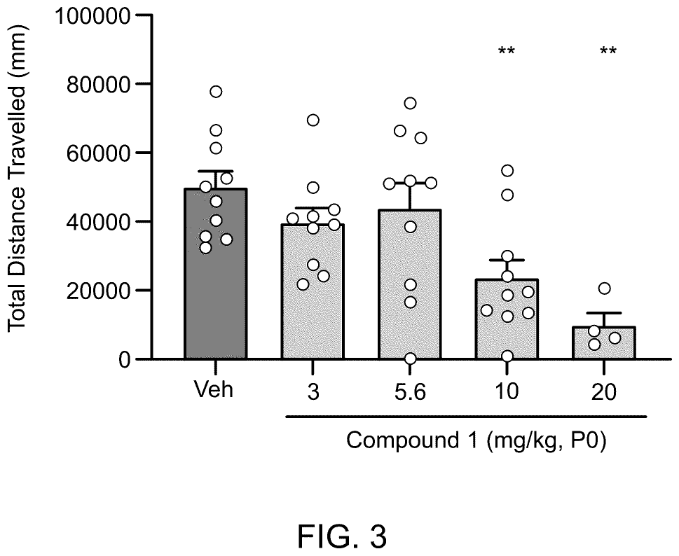

As shown in FIG. 3, Compound 1 at doses of 10 and 20 mg/kg significantly reduced total traveling distance in the 30-minute sLMA test. Compound 1 has a calculated TD₅₀ of 10.3 mg/kg. These data suggest that Compound 1 at a dose of 10 mg/kg and 20 mg/kg reduces locomotor activity.

### Example 5. Compound 1 has Potent Anticonvulsant Activity with Improved Protective Index Relative to Standard of Care Sodium Channel Blockers

This study compared the effects of Compound 1 on sodium current (I_{Na}), intrinsic neuronal excitability, and protection from evoked seizures to two standard voltage-gated sodium channel (Na_{V}) blockers to determine whether a preferential persistent I_{Na} inhibitor, such as Compound 1, would exhibit improved preclinical efficacy and tolerability.

Inhibition of I_{Na} was characterized using patch clamp analysis. The effect on intrinsic excitability was measured using evoked action potentials recorded from hippocampal CA1 pyramidal neurons in mouse brain slices. Anticonvulsant activity was evaluated using the maximal electroshock seizure (MES) model, and tolerability was assessed by measuring spontaneous locomotor activity (sLMA). All assays are described in the preceding examples.

As shown in FIG. 1A, Compound 1 potently inhibited ATX-II-induced persistent I_{Na} expressed by wild-type hNa_{V}1.6, similar to Reference Compound but with a different “Na_{V} Fingerprint,” while the two standard Na_{V}-targeting antiepileptic drugs LTG and CBZ exhibited lower potency and no preference for persistent I_{Na} (FIG. 1C (LTG) and FIG. 1D (CBZ)). As shown in FIG. 2, Compound 1 produced dose-dependent protection (increase in latency) of mice against MES-induced tonic hindlimb seizures. Compound 1 (10 mg/kg) completely blocked evoked seizures (MES ED₅₀ 0.67 mg/kg, brain EC₅₀ 67.2 ng/g) without affecting sLMA (TD₅₀ 10.27 mg/kg, plasma TC₅₀ 1123 ng/g). As shown in FIG. 3, Compound 1 at a dose of 10 mg/kg and 20 mg/kg significantly reduced locomotor activity. While CBZ and LTG also provided dose-dependent protection from MES-induced seizures and dose-dependent reductions in sLMA, the ratio of tolerability to efficacy (protective index) was different and full anticonvulsant efficacy was not achieved without reducing sLMA. The protective index was calculated for each molecule by dividing the brain or plasma TC₅₀ for reduction in sLMA by the brain or plasma EC₅₀ for increasing latency to seizures.

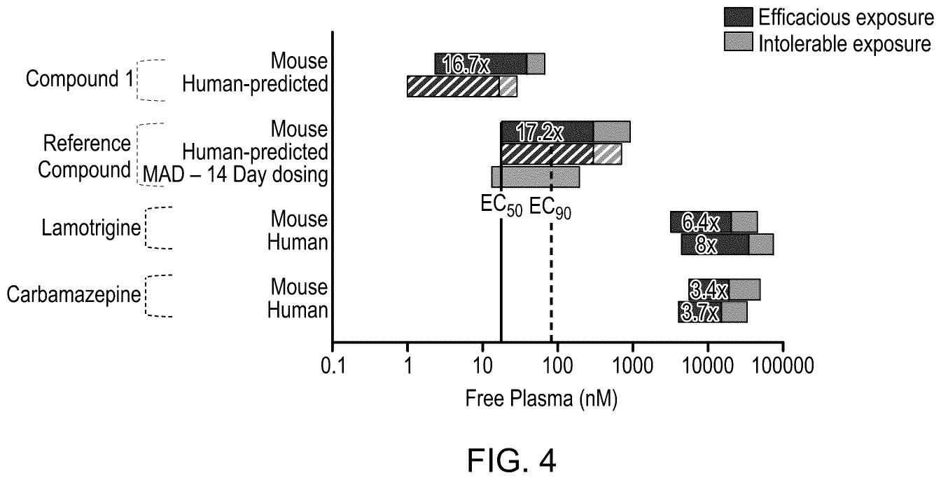

As shown in FIG. 4, Compound 1 (10 mg/kg) had a significantly improved protective index of approximately 16-fold (based on calculated free plasma concentrations), similar to Reference Compound which had an approximately 17-fold (based on calculated free plasma concentrations). This represents an improvement in protective index compared with both CBZ (plasma, 3.4×) and LTG (plasma, 6.4×) as shown in FIG. 4. Thus, Compound 1 exhibited markedly improved preclinical tolerability compared to standard of care Na_{V} blockers. Without intending to be bound by any theory, the improved tolerability of Compound 1 may be due to its demonstrated preference for persistent I_{Na} relative to peak I_{Na} and improved activity dependent inhibition of peak I_{Na}.

### Example 6. Study on Safety, Tolerability, Efficacy, and Pharmacokinetics of Compound 1 in Patients with Focal Epilepsy

Compound 1 mediated blockade of the persistent sodium current (I_{Na}) as shown in animal models can lead to antiseizure efficacy at well tolerated doses. By specifically blocking persistent I_{Na}, Compound 1 can provide greater efficacy in seizure reduction compared to standard of care (SOC) sodium channel blockers (SCB) that are less selective for persistent I_{Na}. Because Compound 1 is less active at peak current, it will be better tolerated than SOC, causing fewer on-target AEs. Accordingly, Compound 1 can be effective and well tolerated when utilized as first line monotherapy allowing for improved patient outcomes and continuity of treatment from infancy to adulthood.

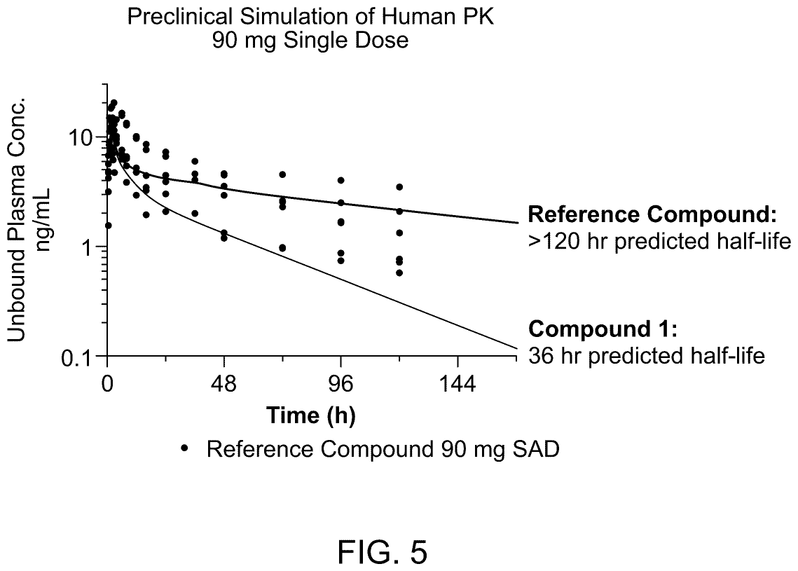

Preclinical data demonstrates that Compound 1 has enhanced selectivity for disease-state Na_{V} channel hyperexcitability and wide therapeutic window, which contributes to its superior safety and efficacy in animal models, and expected therapeutic utility in human patients with epilepsy, such as focal epilepsy. Moreover, as shown in FIG. 5, the predicted half-life of Compound 1 is about 36 hours, allowing acute dosing of Compound 1 in a broader epilepsy patient population. A 28-day pharmacological GLP toxicology study is currently on-going with an aim to understand the onset, degree of severity, and time length up to which a particular dose of Compound 1 demonstrates any toxic effects. A phase 1 clinical study with healthy volunteers with a focus on the Single Ascending Dose/Multiple Ascending Dose (SAD/MAD) studies in a 14-day treatment duration will be conducted, followed by a phase 2 clinical study with focal epilepsy patients to study the safety of Compound 1 as well as its effects in seizure reduction.

### Example 7. Effects of Compound 1 on the Peak Activity of Human Na_{V}1.6 Channels

Voltage-gated sodium channels (Na_{V}) are important therapeutic targets for anti-epileptic drugs (AEDs) due to their role in action potential initiation and propagation. Variants in human Na_{V} genes, such as SCN8A encoding hNa_{V}1.6, are the most common cause of de novo genetic epilepsy and can exhibit gain-of-function profiles leading to neuronal hyperexcitability. Selective Na_{V} blockade during periods of hyperexcitability (activity dependence) has been proposed as a pharmacological target for reducing pathologic neuronal activity, while sparing physiological peak I_{Na} is important to ensuring normal neuronal function. This study investigated the effects of Compound 1 on I_{Na} expressed by hNa_{V}1.6.

### Methods

Persistent and peak I_{Na} inhibition was studied using automated patch clamp recordings of Na_{V} expressed in HEK cells (hNa_{V}1.6). Voltage protocols measured I_{Na} inhibition in multiple modes: persistent I_{Na} (Vm −120 mV, 200 ms), tonic block (TB; Vm −120 mV, 0.2 Hz), voltage-dependent block (VDB; Vm inactivation V_{1/2}), and activity/use dependent block (UDB, Vm −120 mV, 10 Hz). Compound 1 was compared to a panel of AEDs, non-AEDs, and investigational compounds.

Binding kinetics were inferred from inhibition kinetics. The apparent binding rate (K_{ON}) was measured using the time-dependent inhibition of I_{Na} during a variable length conditioning pulse. The apparent unbinding rate (K_{OFF}) was measured using the time-dependent delay in the recovery of I_{Na} following an inactivating conditioning pulse.

### Results

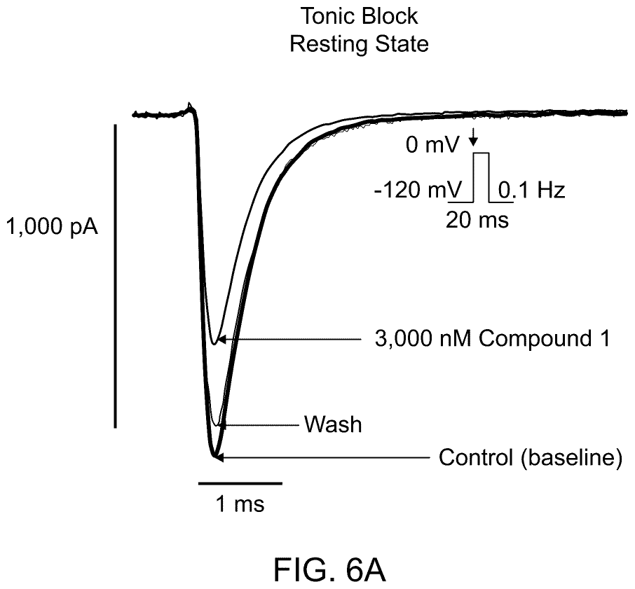

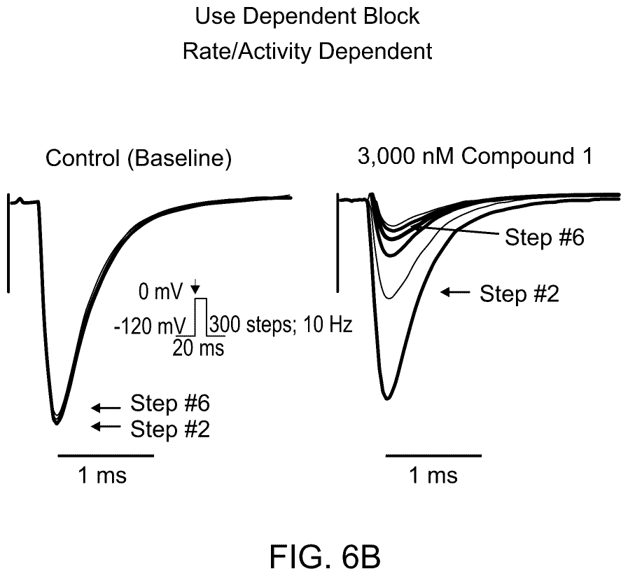

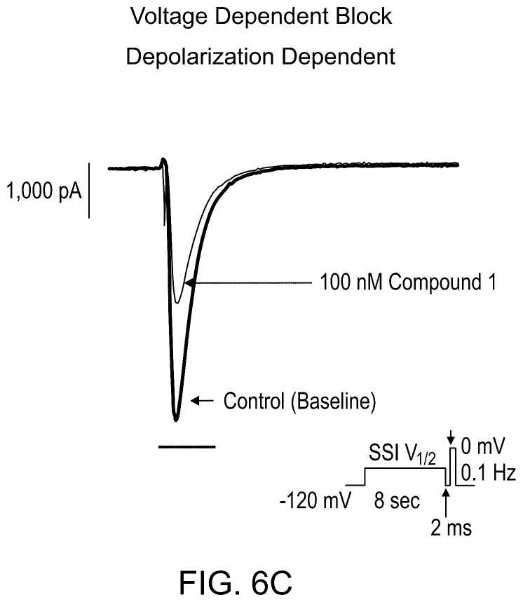

FIG. 6A shows the result of assessment of I_{Na} block using an assay for tonic block in HEK cells.

FIG. 6B shows the result of assessment of I_{Na} block using an assay for use-dependent block in HEK cells.

FIG. 6C shows the result of assessment of I_{Na} block using an assay for voltage-dependent block in HEK cells. Peak I_{Na} was measured at the beginning of the voltage step. FIGS. 6A-6C indicate that Compound 1 exhibited an enhanced activity-dependent block which has been suggested to convey beneficial activity during periods of hyperexcitability.

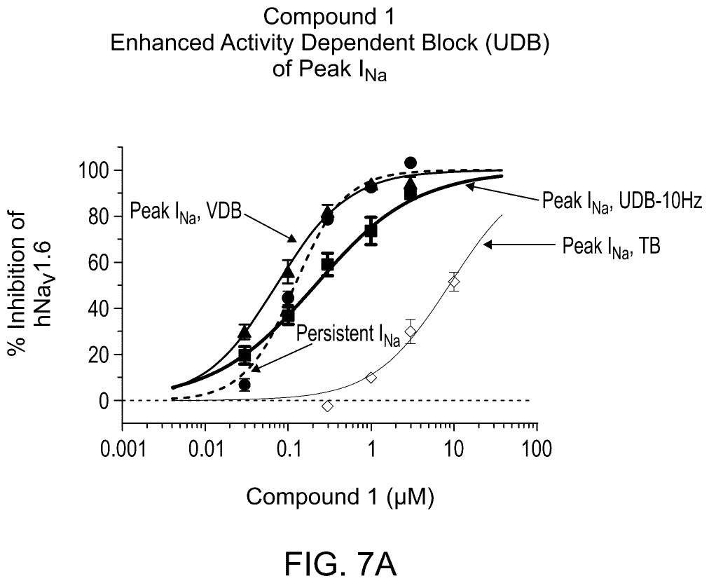

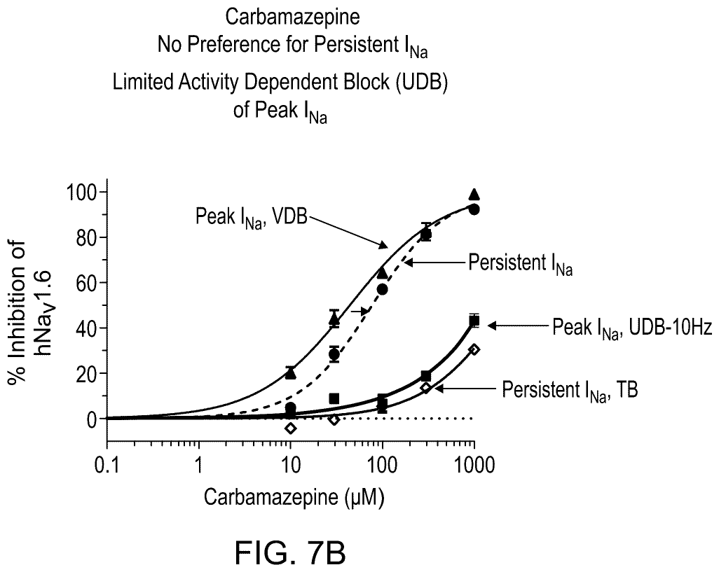

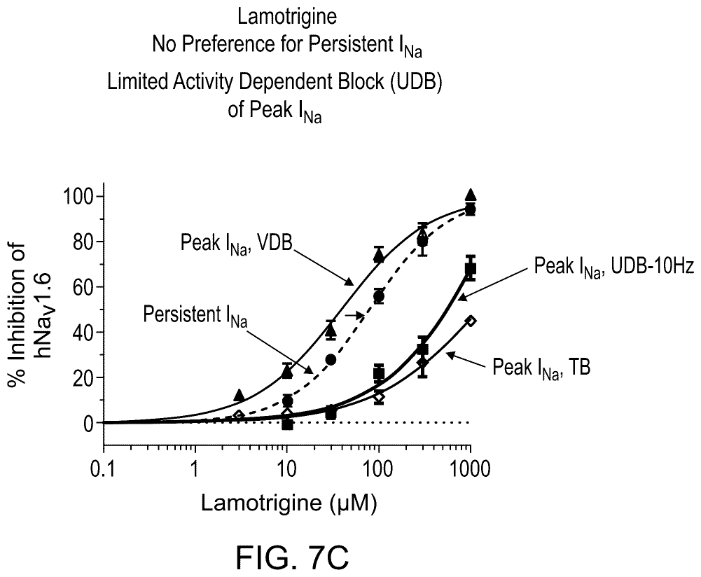

FIG. 7A is a graph showing percent inhibition of hNa_{V}1.6 as a function of the concentration of Compound 1. FIG. 7A demonstrates potent inhibition of persistent I_{Na} and enhanced activity dependent inhibition of peak I_{Na}.

FIG. 7B is a graph showing percent inhibition of hNa_{V}1.6 as a function of the concentration of carbamazepine.

FIG. 7C is a graph showing percent inhibition of hNa_{V}1.6 as a function of the concentration of lamotrigine. FIGS. 7B and 7C demonstrate that carbamazepine and lamotrigine exhibited lower potency and preference for persistent I_{Na} (short arrows) and minimal activity dependent block of peak I_{Na}. Voltage protocols included as panel insets; pharmacology measured at green arrow; points represent mean±SEM.

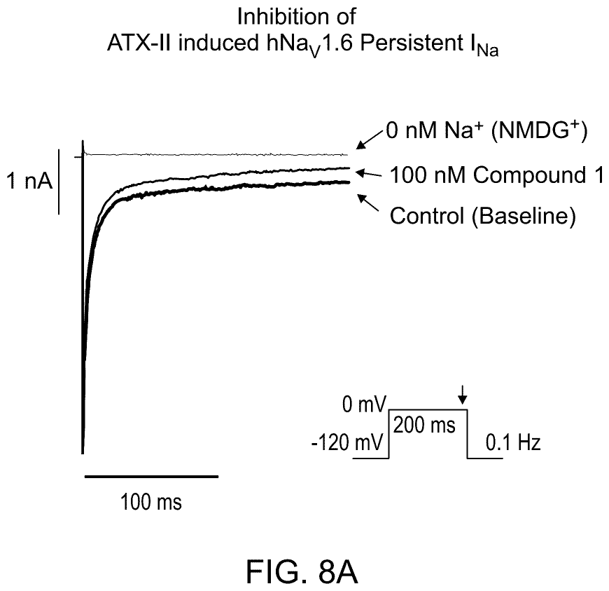

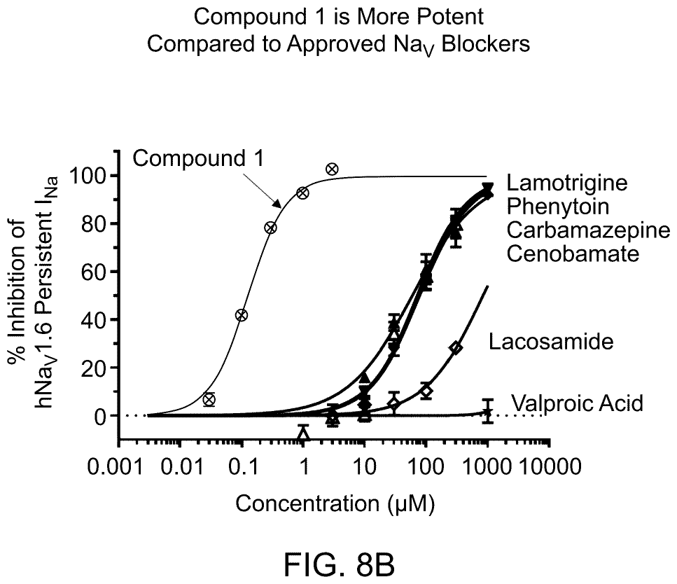

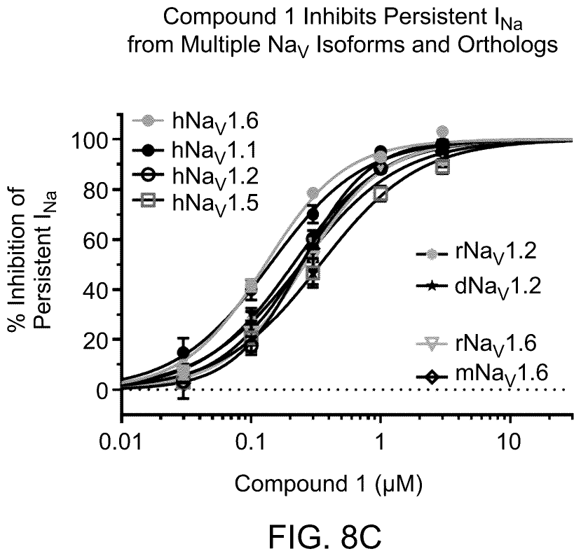

FIG. 8A shows the results of Compound 1-induced reduction in ATX-II evoked hNa_{V}1.6 persistent I_{Na}. Voltage protocol included as panel inset; pharmacology measured at arrow. FIG. 8A indicates that Compound 1 is a potent inhibitor of persistent I_{Na}.

FIG. 8B is a graph showing percent inhibition of hNa_{V}1.6 as a function of the concentration for Compound 1 and standard Na_{V}-targeting ASMs (lamotrigine, phenytoin, carbamazepine, cenbamate, lacosamide and valproic acid). Points represent mean±SEM. FIG. 8B indicates that Compound 1 exhibits increased potency for persistent I_{Na} relative to standard Na_{V}-targeting ASMs.

FIG. 8C is a graph showing the percent inhibition of various Na_{V} isoforms and orthologs as a function of the concentration of Compound 1. Points represent mean±SEM. FIG. 8C indicates that Compound 1 inhibits ATX-II induced persistent I_{Na} expressed by multiple Na_{V} isoforms and orthologs.

The inhibition parameters of hNa_{V}1.6 by Compound 1 and standard Na_{V}-targeting ASMs are shown in the table below. The results presented in the table below indicate that Compound 1 demonstrates greater activity dependence for peak I_{Na} as compared with a panel of standard-of-care Na_{V}-targeting ASMs.

| | | | | Peak I_{Na} | | | |
|---|---|---|---|---|---|---|---|
| IC₅₀, nM | Persistent | Peak I_{Na} | Ratio to | UDB- | Ratio to | Peak I_{Na} | Ratio to |
| (Slope) | I_{Na} | TB | Pers. I_{Na} | 10 Hz | Pers. I_{Na} | VDB | Pers. I_{Na} |
| Compound 1 | 128 | 8,707 | 68 | 200 | 1.6 | 72 | 0.56 |
| | (1.4) | (1.0) | | (0.7) | | (1.0) | |
| | | | | Max 100% | | | |
| Cenobamate | 71,690 | 1,719,000 | 24 | 749,300 | 11 | 66,710 | 0.9 |
| | (1.1) | (1.1) | | (0.7) | | (0.9) | |
| Phenytoin | 59,820 | n/a** | — | 876,600 | 15 | 47,780 | 0.8 |
| | (0.8) | | | (0.6) | | (1.0) | |
| Carbamazepine | 77,490 | 2,307,000 | 30 | 1,418,000 | 18 | 44,370 | 0.6 |
| | (1.1) | (1.0) | | (0.9) | | (0.9) | |
| Oxcarbazepine | 123,700 | 1,035,000 | 8 | n.d. | — | 42,000 | 0.3 |
| | (1.0) | (1.7) | | | | (1.1) | |
| Lamotrigine | 78,480 | 1,249,000 | 16 | 515,800 | 6.6 | 39,090 | 0.5 |
| | (1.0) | (0.8) | | (1.0) | | (0.9) | |
| Lacosamide | 832,700 | n/a** | — | 682,200 | 0.8 | 269,300 | 0.3 |
| | (0.9) | | | (1.3) | | (1.2) | |
| Valproic acid | 2% @ | 11 @ | — | 8% @ | — | 18% @ | — |
| | 1 mM | 1 mM | | 1 mM | | 1 mM | |

Data are IC₅₀ (nM) with the Hill slope in parentheses;
**could not be determined due to compound solubility limit; n.d. = not determined; Pers. = persistent; TB = tonic block; UDB = use-dependent block; VDB = voltage-dependent block.

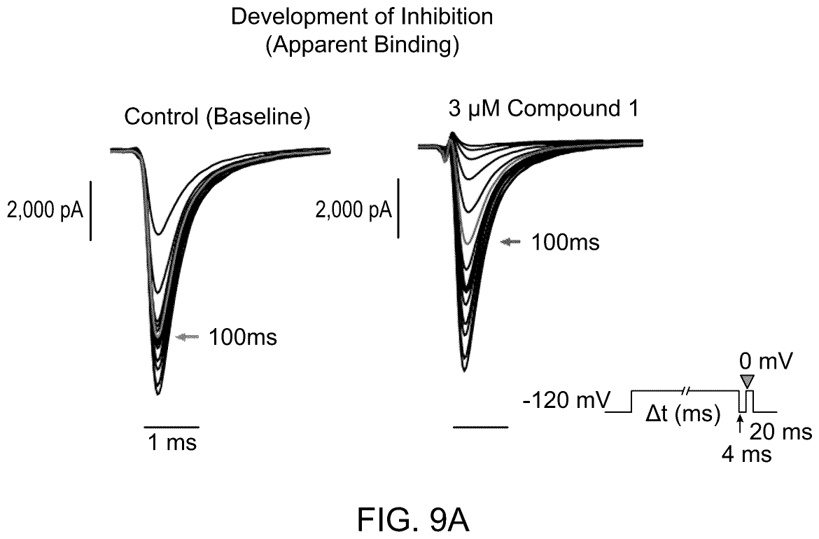

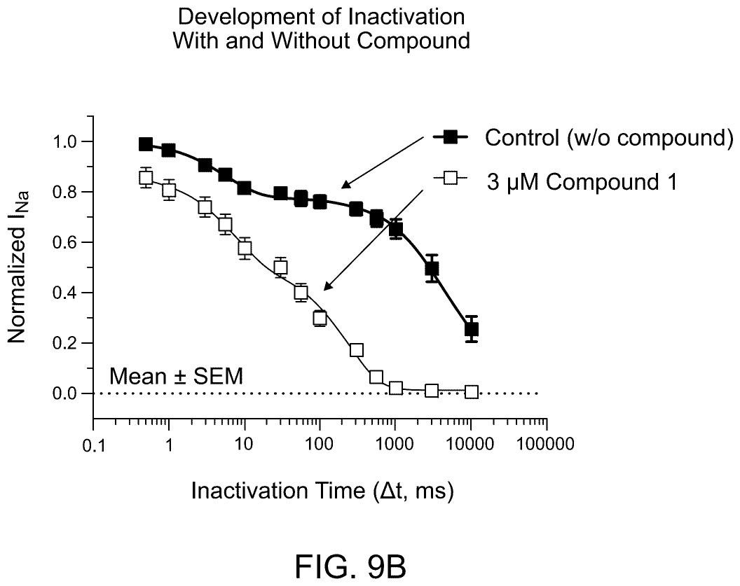

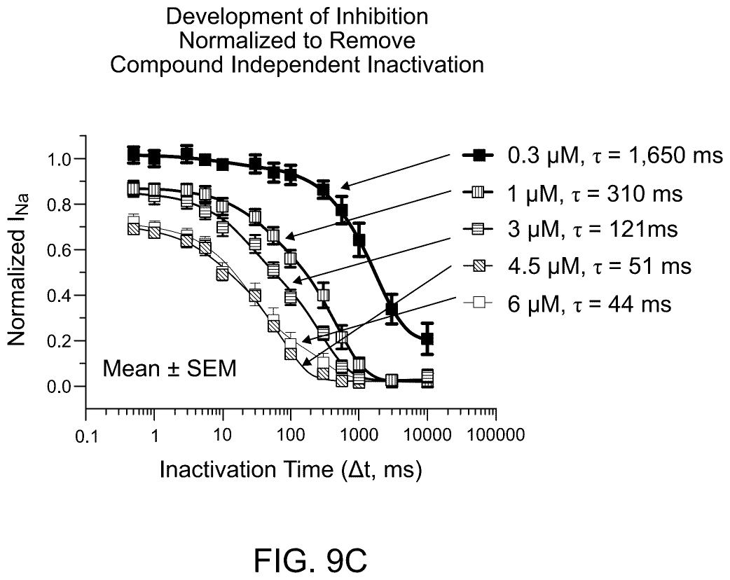

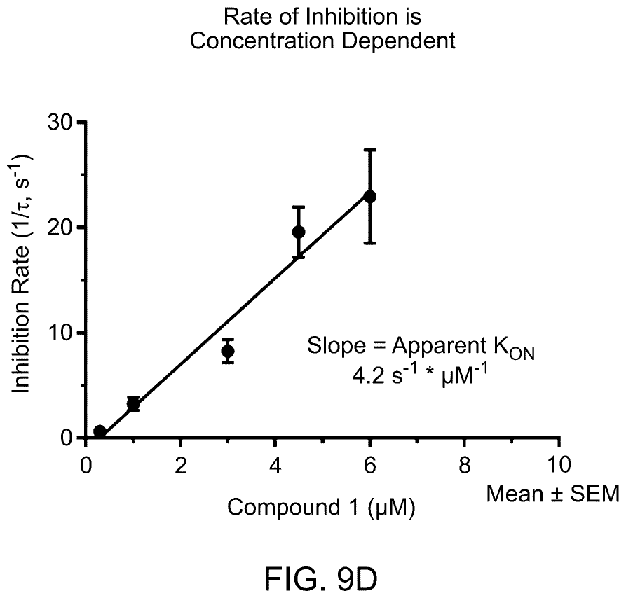

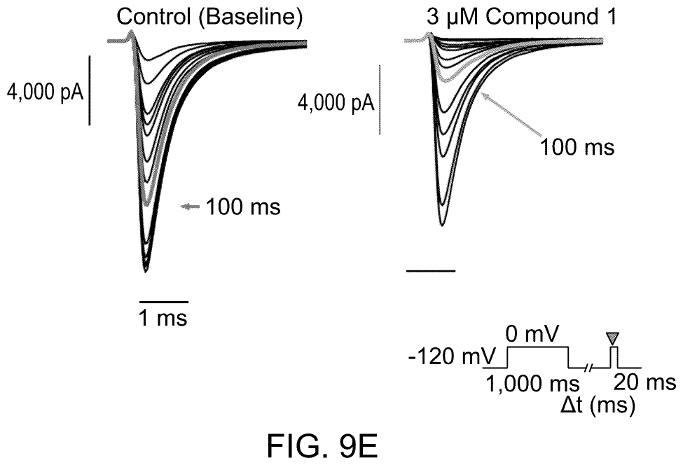

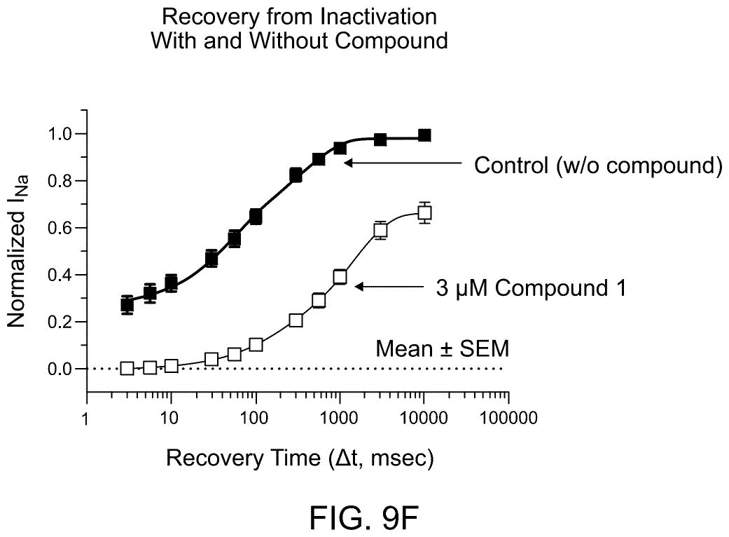

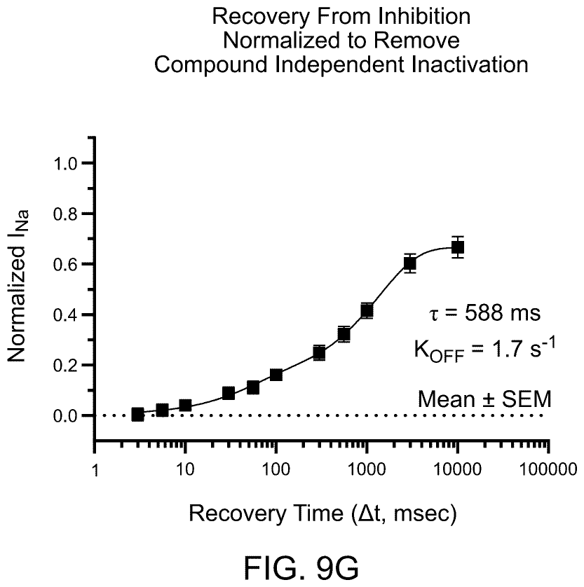

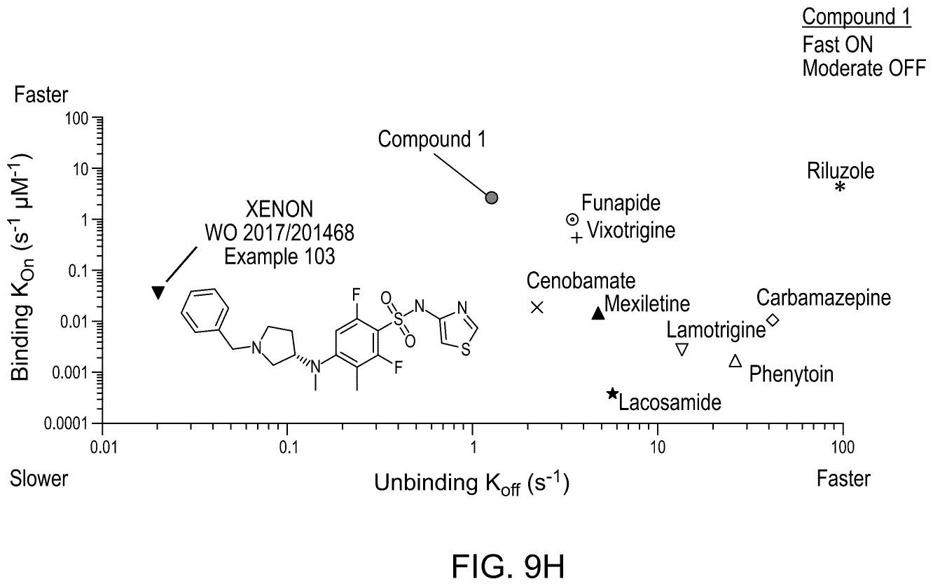

FIG. 9A shows development of inhibition (apparent binding) for 3 μM Compound 1 and control.

FIG. 9B is a graph showing normalized I_{Na} as a function of inactivation time and illustrates the development of inactivation in the absence and presence of 3 μM Compound 1.

FIG. 9C is a graph showing normalized I_{Na} as a function of inactivation time and illustrates the development of inhibition in the presence of Compound 1 at the concentrations of 0.3 μM, 1 μM, 3 μM, 4.5 μM, and 6 μM.

FIG. 9D is a graph showing inhibition rate as a fuction of the concentration of Compound 1 and illustrates that the apparent K_{ON} for Compound 1 is 4.2 s⁻¹*μM⁻¹.

FIG. 9E shows recovering from inhibition (apparent unbinding) for 3 μM Compound 1 and control.

FIG. 9F is a graph showing normalized I_{Na} as a function of recovery time and illustrates recovery from inactivation in the absence and presence of 3 μM Compound 1.

FIG. 9G is a graph showing normalized I_{Na} as a function of recovery time and illustrates recovery from inhibition (normalized to remove compound independent inactivation) and that K_{OFF} for Compound 1 is 1.7 s⁻¹.

FIG. 9H is a graph showing binding K_{ON} and unbinding K_{OFF} for Compound 1 and standard-of-care Na_{V}-targeting ASMs. FIG. 9H indicates that Compound 1 exhibits fast apparent binding and moderate apparent unbinding as compared to standard-of-care Na_{V}-targeting ASMs. The K_{ON} for Compound 1 was fast compared to the other tested compounds (>1,500× versus carbamazepine). The K_{OFF} for Compound 1 was slower than carbamazepine (33×) suggesting a longer, but not excessive residence time. A representative hNa_{V}1.6 isoform selective inhibitor exhibits a very slow unbinding rate (2,079× slower than carbamazepine).

The results presented in FIGS. 9A-9H illustrate that the increased potency and activity dependence of Compound 1 may originate from the rapid development of inhibition (apparent K_{ON}=4.2 s⁻¹*μM⁻¹) paired with a moderate dissociation rate (apparent K_{OFF}=1.7 s⁻¹). Without wishing to be bound by a specific theory, it is believed that the combination of rapid development and recovery from inhibition may selectively target pathological neuronal hyperexcitability, while sparing physiological activity.

### Conclusions

Compound 1 exhibited potent activity dependence (UDB IC₅₀ of 200 nM, 44× preference to TB) which has been suggested to convey beneficial activity during periods of hyperexcitability. Compound 1 also blocked hNa_{V}1.6 persistent I_{Na} with an IC₅₀ of 128 nM (68× preference to TB), which was at least 550× more potent than the other tested I_{Na} blocking agents. This profile was different than that of CBZ (persistent I_{Na} IC₅₀ of 77,490 nM, 30× preference to TB, no UDB observed) and cenobamate (persistent I_{Na} IC₅₀ of 71,690 nM, 24× preference to TB, UDB 2.3× preference to TB). A similar profile was observed at other tested isoforms and orthologs.

The preference for persistent I_{Na} exhibited by Compound 1 was not retained when compared to activity in the more depolarized VDB assay (0.56× preference to VDB); contrasting with a preferential persistent I_{Na} inhibitor (2.2× preference to VDB), aka, Reference Compound.

The enhanced activity dependence of Compound 1 derives from a rapid K_{ON} (4.168 s⁻¹*μM⁻¹) and moderate K_{OFF} (1.72 s⁻¹). This kinetics explains the rapid development of peak I_{Na} relative to standard I_{Na} blockers such as CBZ and cenobamate (K_{ON}s of 0.011 and 0.019 s⁻¹*μM⁻¹, respectively).

Compound 1 is a next generation Na_{V} blocker with increased potency and activity dependence for peak I_{Na} as well as greater potency for persistent I_{Na}. The profile of Compound 1 may translate to efficacy in epilepsy, and other indications caused by neuronal hyperexcitability, without tolerability issues caused by excessive tonic block of peak I_{Na}.

### Example 8. Effect of Compound 1 on Spontaneous Locomotor Activity in Cd-1 Mice

The spontaneous locomotor activity (sLMA) test is a validated model for evaluating the potential effects of a compound on motor function. This study evaluated the effect of Compound 1 on locomotor activity of male CD-1 mice. Doses of Compound 1 used were 3 mg/kg, 5.6 mg/kg, 10 mg/kg and 20 mg/kg administered orally (p.o.).

### Test Animals and Treatment

Male CD-1 mice (Vital River, Beijing, China) approximately 6 weeks old were used for the study. Body weights were between 25 g and 35 g at the time of experiment. The mice were housed under controlled conditions (temperature: 20-26° C.; humidity: 40-70%; air exchange rate: 10-15 cycles/hour; 12:12 light-dark cycle with lights on at 5:00 a.m. Food and water were available ad libitum.

Treatment was conducted using Compound 1 and 35% 2-hydroxypropyl-β-cyclodextrin (35% HPBCD, used as a negative control). Compound 1 stock solution was prepared fresh each experiment day in 35% HPBCD and protected from light. The stock solution was diluted with vehicle for lower doses. Both compound and vehicle were dosed at 10 ml/kg.

### Study Protocol

The spontaneous locomotor activity (sLMA) test for each study was carried out over three days, with mice from each treatment group tested each day. The treatment groups included mice dosed with vehicle (35% HPBCD) and with Compound 1 at the dose of 3 mg/kg, 5.6 mg/kg, 10 mg/kg and 20 mg/kg. Mice were randomly assigned into one of the treatment groups. Animals were acclimated to the test room at least 30 minutes prior to the experiment.

A single dose of Compound 1 (3 mg/kg, 5.6 mg/kg, 10 mg/kg and 20 mg/kg) or vehicle (35% HPBCD) was administered to mice via oral gavage 30 minutes prior to testing. Immediately prior to conducting the sLMA test, a brief neurological assessment was performed. Each animal was visually assessed for sedation and ataxia. Sedation assessment included assessment based on the following categories: a) no sedation (animal exhibits normal locomotor behavior; b) mild sedation (animal shows less locomotion or immobility when alone in its home cage but shows normal locomotor activity if provoked by touching by an observer; c) moderate sedation (animal shows immobility when alone in its home cage and reduced locomotor activity when pushed or provoked by touching by and observer; and d) severa sedation (animal completely loses the ability to move).

Mice were then placed in the center of the test arena (40×40×30 cm, 45±5 Lux on the floor) by an experimenter blinded to the treatment group and were allowed to explore uninterrupted for 30 minutes. Activity was recorded by overhead cameras and was analyzed offline. Video tracking analysis software was used to determine total distance travelled in millimeters. Distance travelled was also calculated in 5-minute bins.

At the completion of the study, short videos (˜(10 seconds) were recorded for representative mice from each treatment group showing adverse effects. Mice were anesthetized with CO₂ until absence of pedal withdrawal reflex, whole blood was collected, and brains were harvested from treatment groups. Whole blood (500 μL) was collected in tubes with 10 μL EDTAK2 and stored on wet ice until centrifugation. Whole blood samples were centrifuged at 2000×g for 5 minutes at 4° C. to isolate plasma. Plasma and brain samples were stored at −80° C. and were subsequently analyzed to determine levels of Compound 1.

All statistical analyses were conducted using GraphPad Prism 9.3. Means were combined and standard error was calculated using the Satterthwaite Approximation to combined standard deviations. Dose-response and concentration-response curves for plasma and brain were fit with a four-parameter log function. TD₅₀ and TC₅₀ values were calculated from these fit curves.

### Results

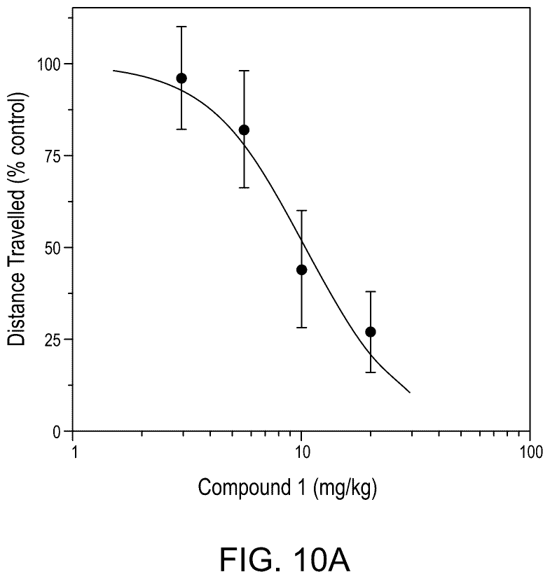

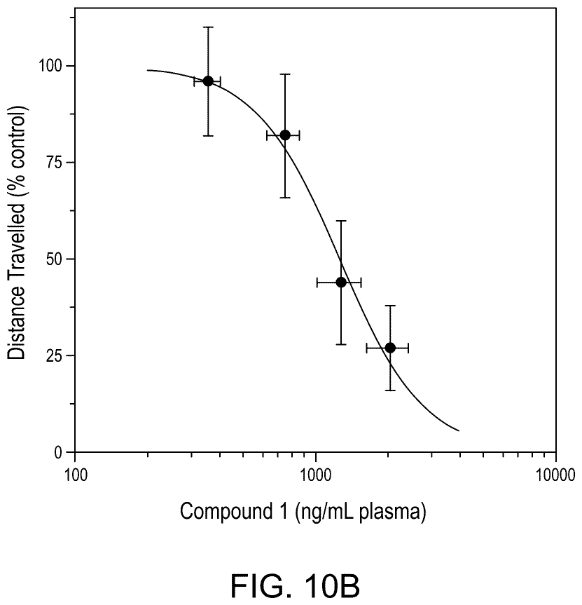

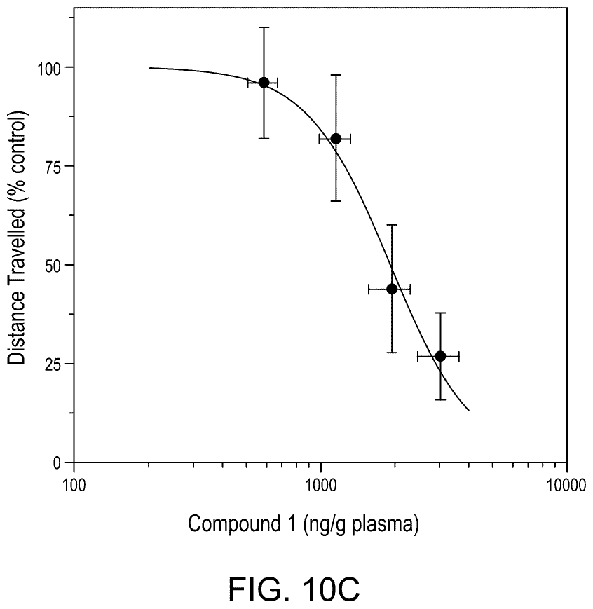

FIG. 10A is a graph showing total distance travelled in the sLMA assay plotted as a percent of control vs. administered dose of Compound 1.

FIG. 10B is a graph showing total distance travelled in the sLMA assay plotted as a percent of control vs. the concentration of Compound 1 in plasma.

FIG. 10C is a graph showing total distance travelled in the sLMA assay plotted as a percent of control vs. the concentration of Compound 1 in the brain. Data are expressed as mean±SEM, with n=20 per group.

The results of the experiment indicate that Compound 1 reduced distance travelled in a dose-dependent manner, with TD₅₀ of 10.4 mg/kg, plasma TC₅₀ of 1256 ng/ml and brain TC₅₀ of 1914 ng/g. No sedation or ataxia was observed for mice treated with Compound 1 at concentrations of 3 mg/kg and 5.6 mg/kg. At higher doses of 10 mg/kg and 20 mg/kg, sedation and/or ataxia was observed in five and eight mice, respectively. Thus, total locomotor activity was reduced at higher doses of Compound 1 (10 mg/kg and 20 mg/kg), a result of ataxia and sedation.

### Example 9. Effect of Compound 1 on MES-Induced Seizure in Male CD-1 Mice

The maximal electroshock (MES) acute seizure model of generalized seizures is validated for evaluating the anticonvulsant potential of compounds. The positive control valproic acid is used clinically for its anticonvulsant properties and is an effective treatment for generalized seizures. The objective of this study was to evaluate the effect of Compound 1 on maximal electroshock induced seizure in male CD-1 mice.

### Test Animals and Treatment

Male CD-1 mice (Vital River, Beijing, China) approximately 6 weeks old were obtained for the study. Body weights were between 20.5 grams and 38.5 grams at the time the experiments were carried out. The mice were housed under controlled conditions (temperature: 20-26° C.; humidity: 40-70%; air exchange rate: 10-15 cycles/hour; 12:12 light-dark cycle with lights on at 5:00 a.m.). Food and water were available ad libitum.

Treatment was conducted using Compound 1 and 35% 2-hydroxypropyl-β-cyclodextrin (35% HPBCD, used as a negative control). Compound 1 stock solution was prepared fresh each experiment day in 35% HPBCD and protected from light. The stock solution was diluted with vehicle for lower doses. Both compound and vehicle were dosed at 10 ml/kg.

### Study Protocol

Mice were brought to the test room at least one hour before the start of the experiment. Mice were administered valproate (VPA; 400 mg/kg, intraperitoneally); or vehicle (35% HPBCD, 10 mL/kg, intraperitoneally); or Compound 1 (0.3 mg/kg, 1 mg/kg, 3 mg/kg or 10 mg/kg, orally) 30 minutes prior to the MES test. Just before the start of the MES test, possible side effects such as overt sedation were recorded. Sedation assessment included assessment based on the following categories: a) no sedation (animal exhibits normal locomotor behavior; b) mild sedation (animal shows less locomotion or immobility when alone in its home cage but shows normal locomotor activity if provoked by touching by an observer; c) moderate sedation (animal shows immobility when alone in its home cage and reduced locomotor activity when pushed or provoked by touching by and observer; and d) severa sedation (animal completely loses the ability to move)

The electroshock apparatus was set to deliver a 50 mA square-wave stimulus, with a 0.8 second duration, a pulse width of 10 milliseconds, and a frequency of 50 Hz. A pair of custom stainless-steel electrodes were soaked in 0.2% agar, and then the subject received bilateral transauricular stimulation through ear-clip electrodes. During the stimulation, mice were manually restrained then released immediately into an observation cage where they were monitored continuously for 60 seconds by an observer blinded to treatment group. Mice were observed for the presence or absence of a generalized tonic-clonic seizure with full hindlimb extension (hindlimbs at a 180 angle to the torso).

Mice were anesthetized with CO₂ and terminal plasma and brain tissue samples were collected. A total of 500 μl whole blood was collected via cardiac puncture and placed in tubes with 10 μl EDTAK2. The tubes were then placed in wet ice until centrifuged at 2,000×g for 5 minutes at 4° C. The supernatant plasma was pipetted into Eppendorf tubes. Both brain and plasma samples were stored at −80° C. and were subsequently analyzed to determine levels of Compound 1.

All statistical analyses were conducted using GraphPad Prism 9.3. Means were combined and standard error was calculated using the Satterthwaite Approximation to combine standard deviations. Dose-response and concentration-response curves for plasma and brain were fit with a four-parameter log function. ED₅₀ and EC₅₀ values were calculated from these fit curves.

### Results

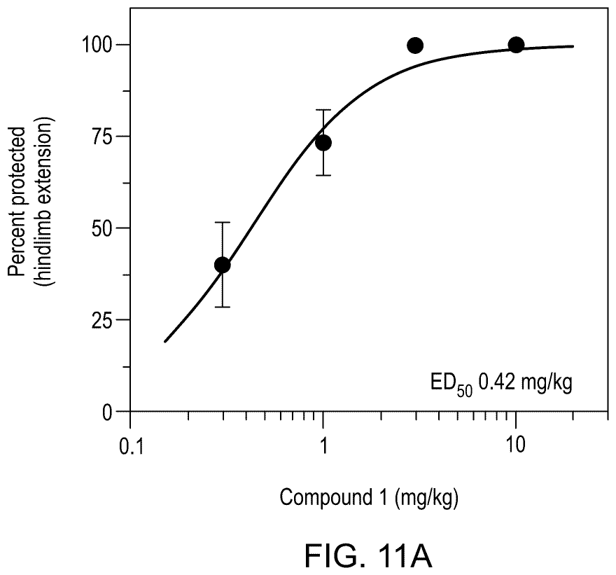

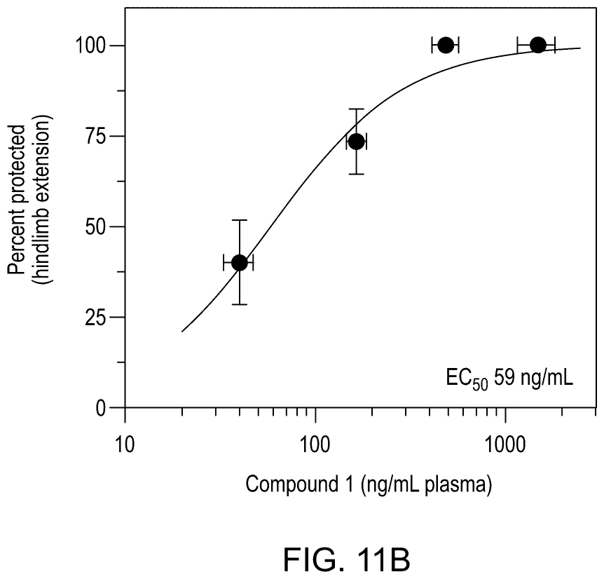

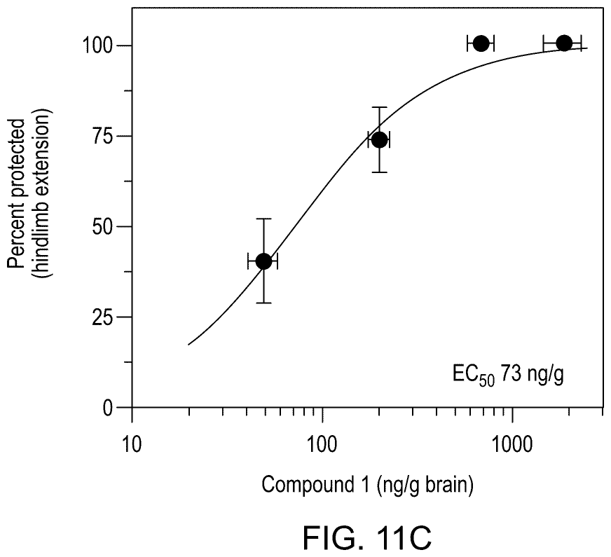

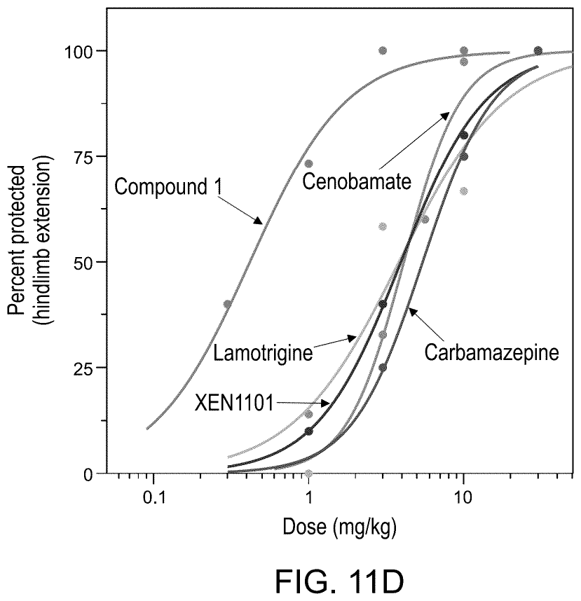

FIG. 11A is a graph showing protection from MES-induced tonic hindlimb extension as a function of the administered dose of Compound 1.

FIG. 11B is a graph showing protection from MES-induced tonic hindlimb extension as a function of the concentration of Compound 1 in plasma.

FIG. 11C is a graph showing protection from MES-induced tonic hindlimb extension as a function of the concentration of Compound 1 in the brain. Data are expressed as mean±SEM, with n=30 per group. Curves represent fit to a four-parameter log function and EC₅₀ values are included in the figures.

As illustrated by FIGS. 11A-11C, protection from MES-induced hindlimb extension by Compound 1 was dose-dependent with a calculated ED₅₀ value of 0.42 mg/kg. The positive control valproate protected against MES-induced hindlimb extension as compared to the negative control (all mice n=24 were seizure-free).

At the highest dose of Compound 1 (10 mg/kg), some mice showed sedation or ataxia. No sedation or ataxia was observed for any of the other doses of Compound 1 tested. Valproate-treated mice showed mild to moderate sedation.

The dose-response curve for protection from MES-induced tonic hindlimb extension for Compound 1 was compared to the dose-response curves for other standard of care anti-seizure mediations (ASMs), i.e., carbamazepine, canobamate, lamotrigine and XEN1101.

FIG. 11D is a graph showing protection from MES-induced tonic hindlimb extension for Compound 1, carbamazepine, cenobamate, lamotrigine and XEN1101 as a function of the administered dose. Curves represent fits to four-parameter log function and error bars have been removed for clarity.

The ED50 values for various compounds tested as shown in the table below.

| Compound | ED₅₀ value (mg/kg) |
|---|---|
| Compound 1 | 0.42 |
| Carbamazepine | 5.4 |
| Cenobamate | 4.1 |
| Lamotrigine | 3.8 |
| XEN1101 | 3.9 |

The results presented in FIG. 11D and the table below indicate that the ED₅₀ value for Compound 1 (0.42 mg/kg) is approximately ten times lower than that of carbamazepine, cenobamate, lamotrigine and XEN1101 (range 3.8-5.4 mg/kg). These results indicate that Compound 1 is active at lower doses as compared to standard of care ASMs in MES acute seizure model.

### Conclusions

Compound 1 acted as an anticonvulsant in the MES acute seizure model as it dose-dependently protected mice against tonic hindlimb extension. No sedation was observed for mice treated with 0.3 to 3 mg/kg of Compound 1. At the highest dose of Compound 1 of 10 mg/kg, six of thirty mice showed mild to moderate sedation, seven and thirteen mice exhibited ataxia with mild to moderate sedation and six mice exhibited ataxia with no sedation. The positive control valproate protected mice from MES-induced tonic hindlimb extension; however, nearly all valproate-treated mice showed mild to moderate sedation.

The results also indicate that Compound 1 is active at lower doses as compared to standard of care ASMs in MES acute seizure model.

### Example 10. Effect of Compound 1 on Pentylenetetrazole (PTZ)-Induced Seizure

The subcutaneous pentylenetetrazole (scPTZ) test is a validated model for evaluating the anti-seizure potential of compounds. The positive control valproate is an approved anti-seizure medicine (ASM). This study evaluated the efficacy of Compound 1 to attenuate scPTZ-induced seizures in wildtype CD-1 mice. Doses of Compound 1 used were 1 mg/kg, 3 mg/kg, 6 mg/kg and 10 mg/kg administered orally. Valproate (600 mg/kg, administered intraperitoneally) was used as a positive control as it has a broad spectrum of anticonvulsant activity.

### Test Animals and Treatment

Male CD-1 mice (Charles River Laboratories, St. Constant, Quebec, Canada) were used for the study. Body weights at the time the experiment was carried out were between 27 and 39 grams. The mice were group-housed in polycarbonate cages according to standard operating procedures. Animals were maintained on a 12 hour light/12 hour dark cycle, with all experimental activities occurring during the light phase. Food and water were available ad libitum. Mice acclimated to the test facility for at least 72 hours prior to testing.

Treatment was conducted using Compound 1 and 35% 2-hydroxypropyl-β-cyclodextrin (35% HPBCD, used as a negative control). Compound 1 stock solution was prepared fresh each experiment day in 35% HPBCD. The stock solution was diluted with vehicle for lower doses. Both compound and vehicle were dosed at 10 ml/kg. Valproate was prepared fresh each experiment day in a vehicle of saline and dosed at 10 ml/kg.

Mice were administered vehicle (35% HPBCD), a single dose of Compound 1 (1 mg/kg, 3 mg/kg or 6 mg/kg) or valproate (600 mg/kg). Vehicle and Compound 1 were administered via oral gavage 30 minutes prior to the administration of PTZ. Valproate was administered as an intraperitoneal injection 60 minutes prior to the administration of PTZ.

### Study Protocol

Immediately prior to the administration of PTZ, a brief neurological assessment was performed. Each animal was visually assessed and received a score from 0 to 3 according to the following scoring system: neurological score of 0 corresponds to a normal status; neurological score of 1 corresponds to a modest decrease in spontaneous activity; neurological score of 2 corresponds to a marked decrease in spontaneous activity; and neurological score of 3 corresponds to loss of righting reflex.

PTZ (85 mg/kg, 10 ml/kg) was administered as a subcutaneous (s.c.) injection. Following the injection of PTZ, each mouse was observed continuously by an observer blinded to treatment until the onset of the first generalized clonic seizure or a time of 30 min was reached. The latency to generalized clonic seizure was recorded. Seizures were also scored using a seizure scale. Mice were scored with the following stages: 0) no seizure activity, 1) myoclonic jerks with “flat body posture”, 2) generalized clonus with loss of posture lasting ˜2 s, 3) generalized clonus with loss of posture lasting ˜10 s or 4) tonic hindlimb extension. Seizure scores were used as a qualitative measure to assess seizure incidence and were not used as a quantitative measure of pharmacological activity. Mice that showed no generalized clonic seizure activity (score of 0 or 1) within 30 min were considered protected. At the end of the PTZ-induced seizure procedure, either at time of generalized clonic seizure or 30 min, plasma and brain samples were collected.

Mice were anesthetized with isoflurane, whole blood was collected via cardiac puncture and brains were harvested. Whole blood was collected in potassium EDTA tubes and centrifuged at ˜3300 g for 5 minutes at 4° C. to isolate plasma. Plasma and brain samples were stored at −80° C. and were analyzed to determine levels of Compound 1.

The experiment was repeated using the study protocol as described above, except for an additional 10 mg/kg treatment group and an adjusted scale used to score seizures. Seizures were scored for using a modified seizure scale. Mice were scored with the following stages: 0) no seizure activity; 1) myoclonic jerks/twitching; 2) myoclonic jerks with “flat body posture”; 3) generalized clonus with loss of posture; or 4) tonic hindlimb extension. Seizure scores were used as a qualitative measure to assess seizure incidence and were not used as a quantitative measure of pharmacological activity. Mice that showed no generalized clinic seizure activity (score of 0, 1 or 2) within 30 minutes were considered protected.

All statistical analyses were conducted using GraphPad Prism 9.3. Means of plasma and brain concentrations were combined and standard error was calculated using the Satterthwaite Approximation to combine standard deviations. Dose-response and concentration-response curves for plasma and brain were fit with a four-parameter log function. ED₅₀ and EC₅₀ values were calculated from these fit curves.

### Results

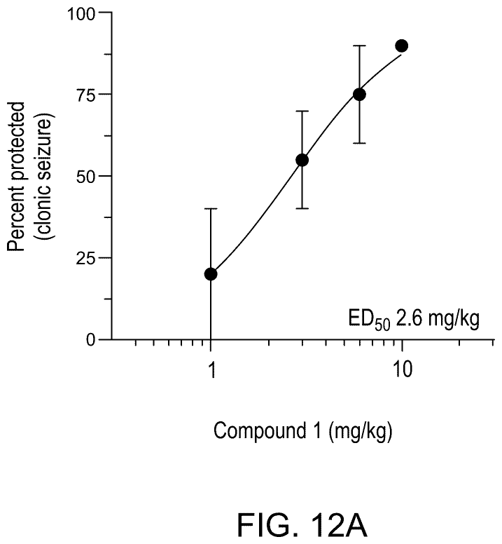

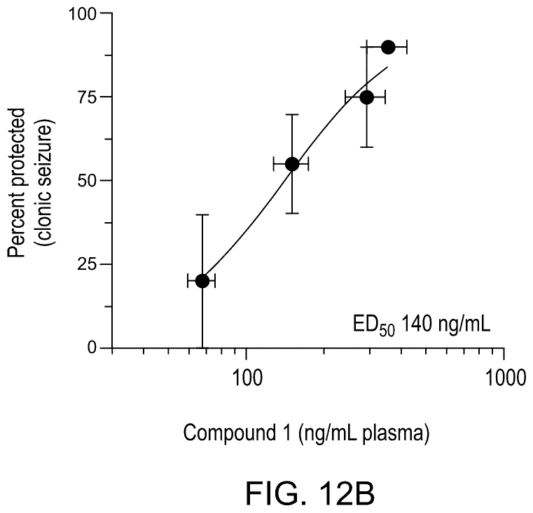

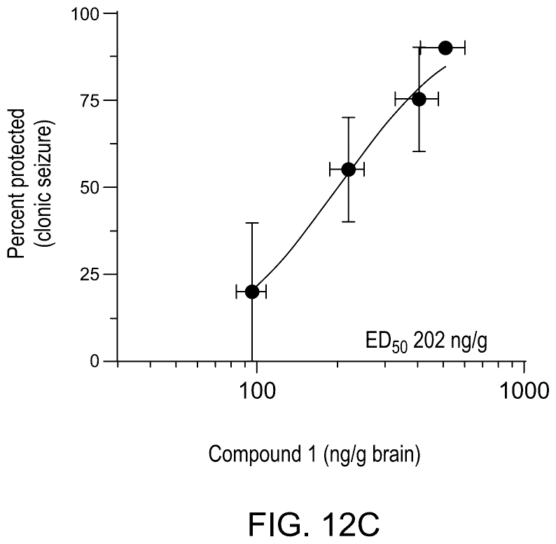

FIG. 12A is a graph showing percent protection from PTZ-induced clonic seizures as a function of the administered dose of Compound 1.

FIG. 12B is a graph showing percent protection from PTZ-induced clonic seizures as a function of the concentration of Compound 1 in plasma.

FIG. 12C is a graph showing percent protection from PTZ-induced clonic seizures as a function of the concentration of Compound 1 in the brain. Data are expressed as mean±SEM, with n=10-20 per group. The curves represent a fit to a four-parameter log function and EC₅₀ values are included in the figures.

### Conclusion

Compound 1 acted as an anticonvulsant in the PTZ acute seizure model as mice were protected from generalized clonus. Neurological scores for all but one mouse treated with Compound 1 were normal, signifying that sedation or any neurological impairment was not observed at the dose range tested. A modest reduction in spontaneous activity was observed in a single mouse treated with 10 mg/kg dose of Compound 1.

### Example 11. Effect of Compound 1 in the 6-Hz Seizure Model

The 6-Hz test is a validated model of focal seizures for evaluating the anti-seizure potential of compounds. The 6-Hz seizure model can be performed at different stimulation intensities (32 or 44 mA), with the higher intensity (44 mA) used as a model of pharmacoresistant seizures. The positive control valproate is an approved anti-seizure medicine (ASM).

This study evaluated the efficacy of Compound 1 in attenuating psychomotor seizures induced by 6-Hz electrical stimulation at two different stimulation intensities (32 and 44 mA) in wild-type CD-1 mice. Psychomotor seizures are defined as the expression of at least one of the following behaviors: stun/immobility, forelimb clonus, straub tail or lateral head movement within 30 seconds of the stimulus. Protection is defined as a complete absence of the above behaviors within 30 seconds of the stimulus. Doses of Compound 1 used were 1 mg/kg, 3 mg/kg and 6 mg/kg administered orally. Valproate (600 mg/kg, administered intraperitoneally) was used as a positive control.

### Test Animals and Treatment

Male CD-1 mice (Charles River Laboratories; St. Constant, Quebec, CAN) were used for the study. Body weights at the time the experiment was carried out were 30-40 grams. The mice were group-housed in polycarbonate cages according to standard operating procedures. Animals were maintained on a 12-hour light/12 hour dark cycle, with all experimental activities occurring during the light phase. Food and water were available ad libitum. Mice acclimated to the test facility for at least 72 hours prior to testing.

Treatment was conducted using Compound 1 and 35% 2-hydroxypropyl-β-cyclodextrin (35% HPBCD, used as a negative control). Compound 1 stock solution was prepared fresh each experiment day in 35% HPBCD. Both compound and vehicle were dosed at 10 mL/kg. Valproate was prepared fresh each experiment day in a vehicle of saline and dosed at 10 mL/kg.

Mice were administered vehicle (35% HPBCD), a single dose of Compound 1 (1 mg/kg, 3 mg/kg or 6 mg/kg) or valproate (600 mg/kg). Vehicle and Compound 1 were administered via oral gavage 30 minutes prior to the administration of PTZ. Valproate was administered as an intraperitoneal injection 60 minutes prior to the electrical stimulus.

### Study Protocol

Immediately prior to conducting the 6-Hz seizure test, a brief neurological assessment was performed. Each animal was visually assessed and received a score from 0 to 3 according to the following scoring system::neurological score of 0 corresponds to a normal status; neurological score of 1 corresponds to a modest decrease in spontaneous activity; neurological score of 2 corresponds to a marked decrease in spontaneous activity; and neurological score of 3 corresponds to loss of righting reflex.

Corneal electrodes moistened with saline (ECT unit 57800; Ugo Basile) were used to deliver the electrical stimulus (6-Hz, 0.2 millisecond pulse width, 3 seconds duration). The current delivered was either 32 mA or 44 mA. Immediately after the electrical stimulus, each mouse was monitored continuously for 30 seconds by an observer blinded to the treatment group. Mice were observed for psychomotor seizure activity defined as stun/immobility, forelimb clonus, Straub tail or lateral head movement. Mice were scored for the presence or absence of each seizure behavior, with a maximum total score of 4. Mice that showed no psychomotor seizure activity within 30 seconds of the electrical stimulus were considered protected and received a score of 0. At the end of the 6-Hz seizure test, plasma and brain samples were collected.

Mice were anesthetized with isoflurane, whole blood was collected via cardiac puncture and brains were harvested. Whole blood was collected in potassium EDTA tubes and centrifuged at ˜3300 g for 5 minutes at 4° C. to isolate plasma. Plasma and brain samples were stored at −80° C. and analyzed to determine levels of Compound 1.

All statistical analyses were conducted using GraphPad Prism 9.3. Seizure scores were compared using Kruskal-Wallis test, followed by Dunn's post hoc and p<0.05 was considered statistically significant.

### Results

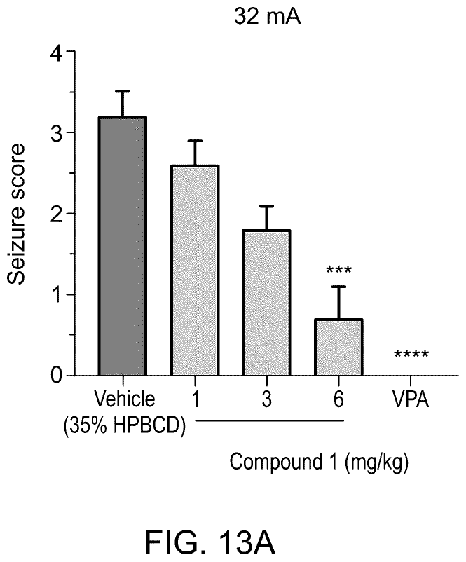

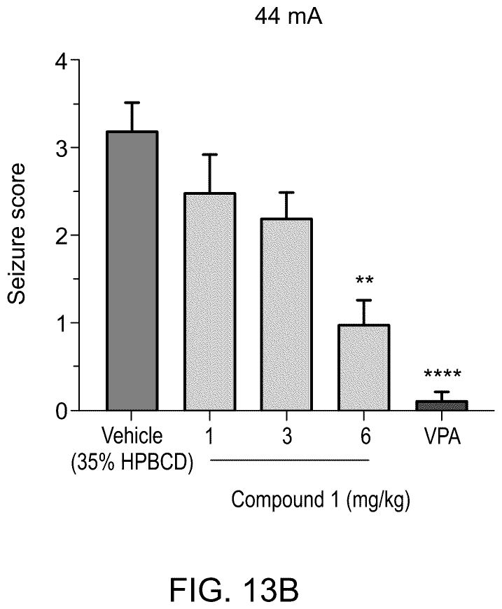

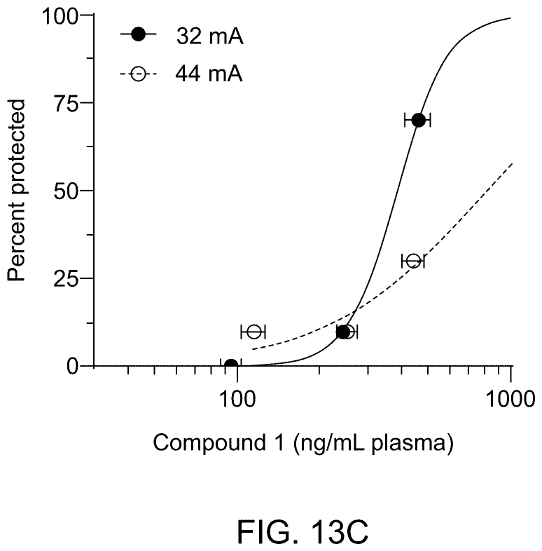

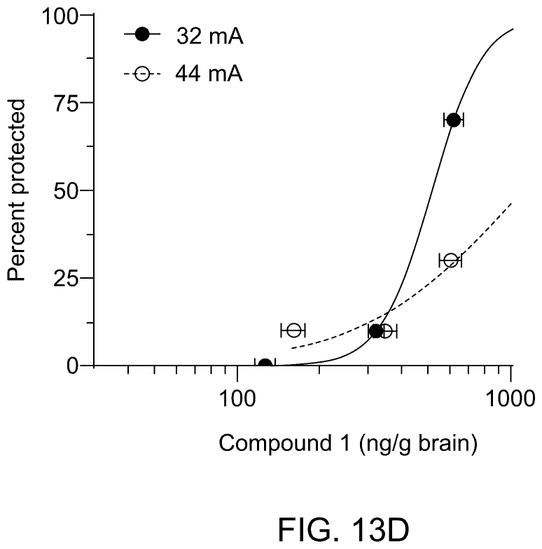

FIG. 13A is a bar graph showing seizure score in the 6-Hz acute seizure model at stimulation current of 32 mA as a function of the administered dose of Compound 1.

FIG. 13B is a bar graph showing seizure score in the 6-Hz acute seizure model at stimulation current of 44 mA as a function of the administered dose of Compound 1.

FIG. 13C is a graph showing percent protection from seizures induced by 6-Hz at stimulation currents of 32 mA (solid symbols, solid line) and 44 mA (open symbols, dashed line) as a function of the concentration of Compound 1 in plasma.

FIG. 13D is a graph showing percent protection from seizures induced by 6-Hz at stimulation currents of 32 mA (solid symbols, solid line) and 44 mA (open symbols, dashed line) as a function of the concentration of Compound 1 in the brain. Data are expressed as mean±SEM, with n=10 per group. The curves represent fit to a four-parameter log function and EC₅₀ values as shown in the table below.

| Current | Plasma EC₅₀ (ng/mL) | Brain EC₅₀ (ng/mL) |
|---|---|---|
| 32 mA | 385 (383-386) | 517 (514-519) |
| 44 mA | >441 | >603 |

Values in parentheses represent the 95% confidence interval.

In addition, dose-response curve for protection from psychomotor seizures was generated using a fit to a four-parameter log function and yielded ED₅₀ value of 1.9 mg/kg. Plasma concentration-response curve for protection from psychomotor seizures was generated using a fit to a four-parameter function and yielded EC₅₀ value of 147 mg/kg.

As is evidenced by the data presented in FIGS. 13A-13D, Compound 1 acted as an anticonvulsant in the 6-Hz psychomotor seizure model. A 6 mg/kg dose administered orally significantly reduced the seizure score at both 32 and 44 mA stimulation currents. Over 50% of the mice treated with the 6 mg/kg of Compound 1 were protected against the 32 mA stimulus. Mice treated with any dose of Compound 1 appeared normal, so all neurological scores were zero. Valproate-treated mice exhibited a modest to marked decrease in spontaneous activity (neurological scores ranged from 1 to 2, average score of 1.2).

### Conclusions

Compound 1 (6 mg/kg, p.o.) was anticonvulsant in the 6-Hz acute seizure model as it significantly reduced the incidence of seizures at the 32 mA current and reduced seizure scores compared to vehicle (negative control) at both 32 and 44 mA stimulation intensities. Specifically, over 50% of mice treated with 6 mg/kg of Compound 1 were protected against the 32 mA stimulus. Neurological scores for all doses of Compound 1 tested were normal, signifying that sedation was not observed. In contrast, valproate-treated mice exhibited a modest to marked decrease in spontaneous activity.

### Example 12. A Phase 1 Clinical Trial to Evaluate the Safety, Tolerability, Pharmacokinetics and Pharmacodynamics of Single and Multiple Ascending Doses of Compound 1 in Healthy Volunteers

This is a trial to assess the safety, tolerability, pharmacokinetics (PK), and pharmacodynamics (PD) of Compound 1 in healthy participants aged 18 to 55 years (inclusive). The trial is initially comprised of 2 parts, with 1 additional optional part, as follows:
Part A is randomized, double-blinded, and placebo-controlled. Part A is designed to investigate the safety, PK, and PD of single ascending doses of Compound 1.
Part B is randomized, double-blinded, and placebo-controlled. Part B is designed to investigate the safety, PK, and PD of multiple ascending doses (selected based on the results from Part A) of Compound 1.
Part C (optional) is a randomized, open-label, crossover design food effect evaluation to investigate the PK of a single dose (selected based on the results from Part A) of Compound 1 in the fasted and fed state.

Part B will commence following the completion of at least Cohort A3 in Part A. Initiation of Part C is optional, based on the results of Parts A and B. Parts A, B, and C may be conducted concurrently. Parts A and B are double blinded. Parts A, B, and C consist of 3 periods: Screening/Baseline, Intervention, and Safety Follow-up.

Safety and tolerability assessments for Parts A, B, and C will include vital signs, 12-lead ECGs, physical examinations, clinical laboratory tests, and the C-SSRS (for Part B).

### Number of Participants

The planned number of participants is as follows:
Part A: Approximately 32, up to 56 participants (8 per cohort)
Part B: Approximately 24, up to 40 participants (8 per cohort)
Part C: Approximately 16 participants

The total number of planned participants across Parts A, B, and C is approximately 72, up to 112 depending on optional cohorts. Participants in Parts A and B may also participate in Part C provided there is sufficient washout between participation in study parts.

Duration of the clinical trial is outlined in the table below.

| Screening/Baseline Period | ≤42 days |
|---|---|
| Intervention Period | Part A: 6 days |
| | Part B: 13 days |
| | Part C: 12 days (+3-day |
| | additional washout, if required) |
| Safety Follow-up Period | Part A: 3 days (±2 days) |
| | Part B: 5 days (±2 days) |
| | Part C: 4 days (±2 days) |
| Total duration of participation | Part A: Up to 53 days |
| | Part B: Up to 62 days |
| | Part C: Up to 63 days |

Objectives and endpoints for Part A of the clinical trial (single ascending dose) is outlined in the table below.

| | |
|---|---|
| Objective | Endpoint |

Primary

| | |
|---|---|
| To assess the safety | Incidence and severity of adverse events (AEs) |
| and tolerability | Changes in vital sign measurements |
| of single oral doses | Changes in clinical laboratory results |
| of Compound 1 | Changes in electrocardiogram (ECG) |
| | parameters |

Secondary

| | |
|---|---|
| To evaluate the | Plasma concentrations of Compound 1 |
| pharmacokinetics (PK) | Maximum observed concentration (Cₘₐₓ) |
| of single oral doses | Time to maximum observed concentration |
| of Compound 1 | (tₘₐₓ) |
| | Area under the drug concentration-time curve |
| | from time zero to infinity (AUC_{inf}) |
| | Area under the concentration time curve from |
| | time zero to the last measurable concentration |
| | (AUCₗₐₛₜ) |
| | Apparent terminal elimination half-life (t_{1/2}) |
| | Clearance (CL/F) |
| | Volume of distribution (Vd/F) |
| | Dose normalized Cₘₐₓ |
| | Dose normalized AUC_{inf} |

Exploratory

| | |
|---|---|
| To evaluate | Changes in quantitative electroencephalography |
| pharmacodynamic (PD) | (qEEG) biomarker(s) (absolute and relative |
| effects of | powers in the delta, theta, beta, alpha and |
| Compound 1 | gamma bands) compared to baseline |
| | Changes in the NeuroCart assessments (other |
| | than qEEG) compared to baseline |

Baseline for PD measurements are defined as the average value prior to dosing in the morning of Day 1 for each treatment period.

Objectives and endpoints for Part B of the clinical trial (multiple ascending dose) is outlined in the table below.

| | |
|---|---|
| Objective | Endpoint |

Primary

| | |
|---|---|
| To assess the safety | Incidence and severity of AEs |
| and tolerability | Changes in vital sign measurements |
| of 10-day repeat | Changes in clinical laboratory results |
| oral doses of | Changes in ECG parameters |
| Compound 1 | Incidence of Columbia-Suicide Severity |
| | Rating Scale (C-SSRS) measured suicidal |
| | ideation or behavior |

Secondary

| | |
|---|---|
| To evaluate the | Plasma concentrations of Compound 1 |
| PK of 10-day repeat | Cₘₐₓ |
| oral doses of | tₘₐₓ |
| Compound 1 | AUCₗₐₛₜ |
| | AUCₜₐᵤ |
| | t_{1/2} |
| | Accumulation ratio based on AUC |
| | (Rac(AUC)) |
| | Accumulation ratio based on Cₘₐₓ |
| | (Rac(Cₘₐₓ)) |

Exploratory

| | |
|---|---|
| To evaluate PD | Changes in quantitative electroencephal- |
| effects of | ography (qEEG) biomarker(s) (absolute |
| Compound 1 | and relative powers in the delta, theta, |
| | beta, alpha and gamma bands) compared |
| | to baseline |
| | Changes in the NeuroCart assessments |
| | (other than qEEG) compared to baseline |
| To evaluate the | Plasma and urine metabolite profile |
| metabolite profile of | |
| COMPOUND 1 following | |
| repeat oral doses | |

Baseline for PD measurements is defined as the average value prior to dosing in the morning of Day 1 for each treatment period.

Objectives and endpoints for Part C of the clinical trial (optional food effect) is outlined in the table below.

| | |
|---|---|
| Objective | Endpoint |

Primary

| | |
|---|---|
| To assess the safety and | Incidence and severity of AEs |
| tolerability of oral | Changes in vital sign measurements |
| doses of Compound 1 | Changes in clinical laboratory results |
| in the fed and fasted | Changes in ECG parameters |
| state | |

Secondary

| | |
|---|---|
| To evaluate the effect | Plasma concentrations of Compound 1 |
| of food on the PK of | Cₘₐₓ |
| Compound 1 | tₘₐₓ |
| | AUC_{inf} |
| | AUCₗₐₛₜ |

### Screening/Baseline Period

The Screening period for all 3 parts will be up to 42 days in duration (Day −43 to Day −2). Prior to any clinical trial procedures, participants will provide written informed consent to participate in the trial. A full medical screening will be performed to assess a subject's eligibility for this study. Following confirmation of continued eligibility, participants will check-in to the clinic at Baseline (Day −1; the day before study drug administration).

### Intervention Period

In Parts A and B participants will remain in the unit from Baseline (Day −1) to Discharge (Day 3 in Part A, Day 11 in Part B). In Part C, participants will remain in the unit from Baseline (Day −1) to Day 3 (first dosing period) and Day 6 to Day 9 (second dosing period). In Part A, participants will return to the clinical research unit for safety and PK assessments on Day 4 and Day 6. In Part B, participants will return to the clinical research unit for safety and PK assessments on Day 13. In part C, participants will return to the clinical research unit for safety and PK assessments on Day 4 and 10. If participants experience any clinically significant AEs during the confinement period, they may remain in the clinical facility for further observation at the discretion of the principal investigator (PI), following consultation with the sponsor.

### Part A (Single Ascending Dose)

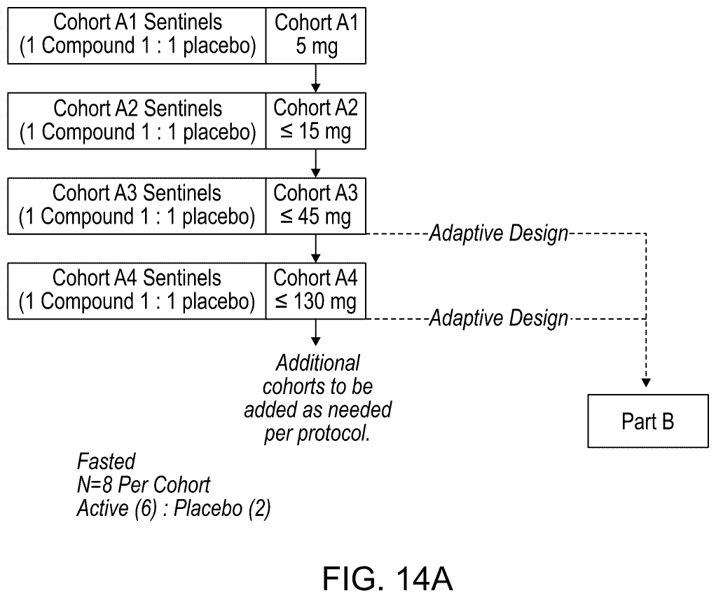

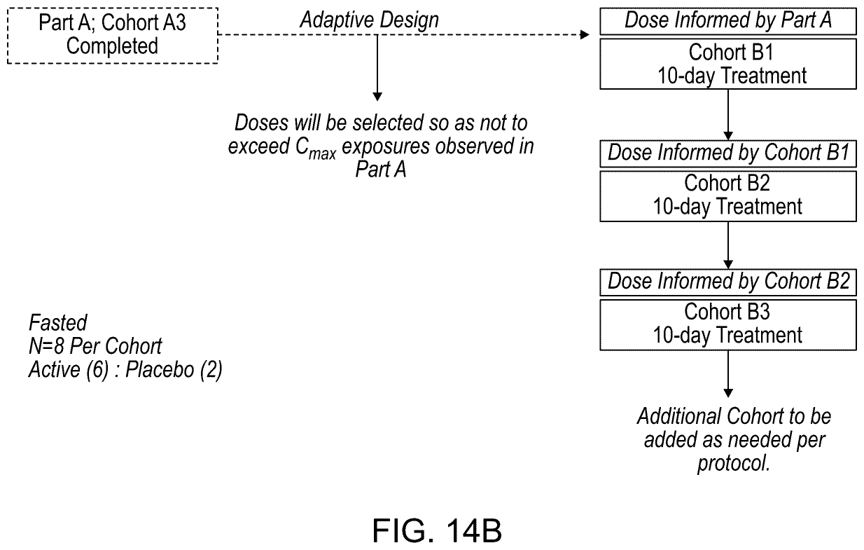

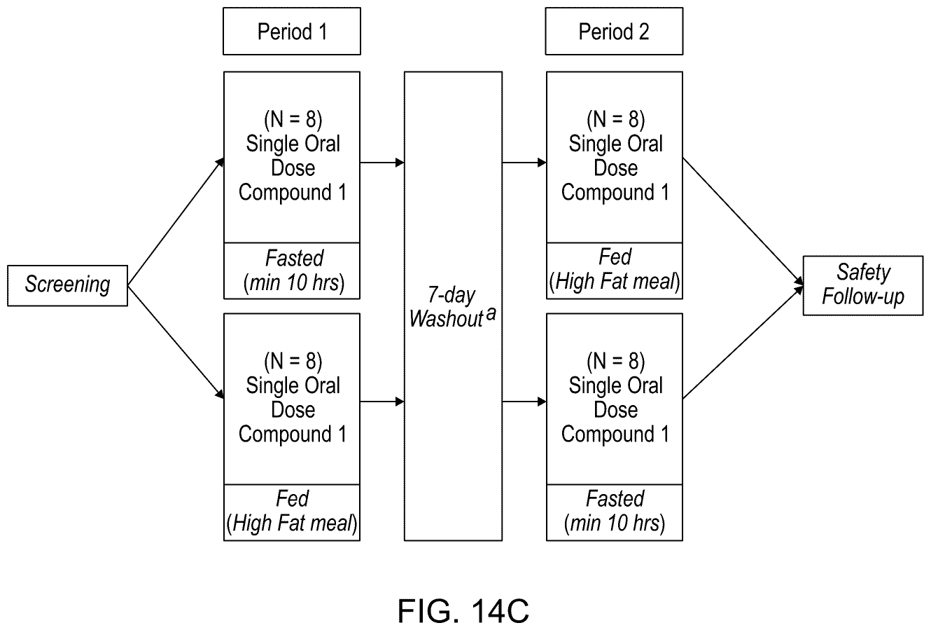

Healthy participants will be enrolled to receive single ascending oral doses of Compound 1 or placebo on Day 1. FIG. 14A is an illustration of the dosing scheme for Part A (single ascending dose) of the trial. Dose escalation in Part A will initially be conducted in up to a total of 4 planned cohorts (Cohorts A1 to A4). Following completion of Cohort A4, up to an additional 3 escalation cohorts may be added. Increment-based escalation will be used in these additional 3 cohorts, with a maximum increment of 3-fold the highest prior dose tested (note: if the projected concentrations of Compound 1 are to exceed 415 ng/ml, the mouse sLMA EC₅₀, a measure of tolerability, the dose increase increment will be ≤1.5-fold). Doses may be adjusted upward or downward based on safety, tolerability, and PK data from the preceding cohorts. Cohorts after A4 will be numbered sequentially (A5, A6, etc.). Dose escalation will be stopped if the mean AUC_{inf} of the next dose is predicted to exceed an AUC_{inf} of 7500 hr.ng/ml (half of the rat NOAEL) unless otherwise justified via a substantial amendment. Eight participants will be enrolled in each cohort and will be randomized to receive either Compound 1 or placebo in a 3:1 ratio.

The dosing regimen for Part A is summarized in the table below.

| Cohort | Study Drug | Dose and Route of Administration |
|---|---|---|
| A1 | Compound 1 | 5 mg, oral, fasted |
| A2 | Compound 1 | Up to 15 mg, oral, fasted |
| A3 | Compound 1 | Up to 45 mg, oral, fasted |
| A4 | Compound 1 | Up to 130 mg, oral, fasted |
| A5 and | Compound 1 | Maximum of 3-fold over of the highest |
| beyond | | prior dose tested, oral, fasted |

Compound 1 will be administered to Cohort A1 participants at the starting dose of 5 mg. Dosing in all cohorts will start with 2 sentinel participants with 1 of the 2 participants randomized to receive Compound 1 and the other participant randomized to receive placebo. The safety and tolerability of each sentinel participant will be monitored until Day 2 and will be reviewed prior to dosing the remainder of participants in the cohort. The PI will review safety/tolerability information available on the sentinel participants on Day 2 and, with agreement from the sponsor, will make the decision to dose the remaining 6 participants in the cohort.

Cohorts will be dosed in an escalating order. After at least 6 out of 8 participants in each dose cohort has completed dosing, blinded cumulative safety data collected up to Day 4 (72 hours post-dose) and available blinded PK data will be reviewed by the safety review committee (SRC) to determine the safety and tolerability of the study drug. If the current dose level is determined to be safe and tolerated by the SRC, the next dose cohort will be randomized to receive the selected dose of active Compound 1 or placebo. Additional cohorts may be considered to accommodate dose repetition, dose reduction, or slower dose escalation than planned.

### Part B (Multiple Ascending Dose)

Following evaluation of Compound 1 administered as a single dose through cohort A3 at a minimum in Part A, the study will commence evaluation of the safety, tolerability, PK and PD of Compound 1 in a multiple dosing schedule. FIG. 14B is an illustration of the dosing scheme for Part B (multiple ascending dose) of the trial. The starting dose level in Part B will be determined based on safety, tolerability, and PK data obtained in Part A. Compound 1 dose levels to be evaluated in Part B will not exceed doses evaluated in Part A.

Cohorts will be numbered sequentially (B1, B2, etc.) with a minimum of 3 cohorts and up to 2 additional cohorts (a maximum of 5 cohorts). Eight participants will be enrolled in each cohort and will be randomized to receive either Compound 1 or placebo in a 3:1 ratio.

The dosing regimen for Part B is summarized in the table below.

| Cohort | Study Drug | Dose and Route of Administration |
|---|---|---|
| B1 | Compound 1 | Dose informed by Part A, oral, fasted |
| B2 | Compound 1 | Dose informed by Cohort B1, oral, fasted |
| B3 | Compound 1 | Dose informed by Cohort B2, oral, fasted |
| B4 and | Compound 1 | Dose(s) found safe and tolerable in Part A, |
| beyond | | or previously studied doses, or intermediate |
| | | dose, oral, fasted |

Healthy participants will be enrolled to receive multiple ascending oral doses of Compound 1 or placebo once daily. Dosing will commence on Day 1 and will continue until Day 10. The last dose will be administered on the morning of Day 10. Participants will be discharged from the clinical research unit 24 hours after the last dose administration.

After at least 6 out of 8 participants in each dose cohort in Part B have been dosed through Day 10, the blinded safety data (including safety assessments performed on Day 10) and available blinded PK data will be reviewed to determine the safety and tolerability of the study drug. If the current dose level is determined to be safe and tolerated, the next dose cohort will be randomized to receive the selected dose of active Compound 1 or placebo. Additional cohorts may be considered to accommodate dose repetition, dose reduction, or slower dose escalation than planned. In addition, the SRC may also consider additional cohorts in the event that the maximum exposure has not been reached with any of the planned dose levels and no stopping criteria have been met.

### Part C (Optional Food Effect Evaluation)

Initiation of Part C is optional, based on the results of Parts A and B. Part C may commence once the safety and PK of Compound 1 have been adequately evaluated in Part A (note: Parts A, B and C may be conducted concurrently). FIG. 14C is an illustration of the dosing scheme for Part C (optional food effect evaluation) of the trial. Approximately 16 participants will receive 2 oral doses of Compound 1, one dose after a minimum of 10 hours fasting and one after (dosing within 30 minutes) the consumption of a high-fat, high calorie meal in a randomized crossover design. Participants will stay in the clinical research unit from Day −1 to Day 3 for each separate dosing visit with a 7 day wash out. The 7-day wash out period is dependent on the PK data of Part A and B. Additional days of washout may be added between fasted and fed dosing based on the observed half-life in Part A. The dose to be used in Part C will not exceed 50% of the maximum dose achieved in Part A. Part C is not blinded and not placebo controlled.

### Safety Follow-up Period

Part A: Day 9 (±2 days)
Part B: Day 18 (±2 days)
Part C: Day 16 (±2 days)

### Number of Participants

Eight participants per cohort are planned in Parts A and B. The total number of participants required will depend upon the number of escalation steps. The clinical trial is anticipated to include approximately 72, or up to 112 participants across all 3 parts (Parts A, B, and C). In Part A, approximately 32, or up to 56 participants are planned to be administered Compound 1 or placebo. In Part B, approximately 24, or up to 40 participants are planned to be administered Compound 1 or placebo. In Part C, up to 16 participants are planned to be administered Compound 1.

### Clinical Trial Intervention

In part A of the trial, on the dosing day, a single ascending dose of Compound 1 (starting at 5 mg) or matching placebo will be administered orally (fasted) and provided in single dose containers to the participants.

In part B of the trial, on all dosing days, multiple ascending doses of Compound 1 (starting at a dose selected from Part A and up to the maximum dose studied in Part A) or matching placebo will be administered once daily orally (fasted) and provided in single dose containers to the participants.

In part C of the trial, on all dosing days, a single dose of Compound 1 (dose selected from Part A) will be administered orally (either fasted or fed) and provided in single dose containers to the participants.

### Preliminary Findings

Compound 1 was observed to be safe and generally well tolerated in healthy participants at all single doses up to 45 mg tested in Part A of the trial. All treatment-emergent adverse events (TEAEs) reported thus far have been mild in severity and resolved without administration of concomitant medication. No severe TEAEs were reported. The most frequently reported TEAEs were headache and fatigue. No deaths or TEAEs leading to study drug discontinuation have been reported. No safety findings were observed with regards to clinical laboratory test results, electrocardiograms (ECGs), and vital signs.

### Example 13. A Phase 2 Trial to Evaluate the Photoparoxysmal Electroencephalogram Response, Safety, Tolerability, and Pharmacokinetics of Compound 1 in Participants with Epilepsy and a Photoparoxysmal Electroencephalogram Response to Intermittent Photic Stimulation

Compound 1 is a novel inhibitor of persistent and peak sodium current (I_{Na}) that is differentiated from currently available sodium channel (Na_{V}) blockers and is being developed for the treatment of adult focal onset epilepsy. Focal epilepsy is characterized by localized (focal) areas of neuronal/network hyperexcitability that disrupts normal brain function by causing periods of abnormal network synchronization (seizure). Na_{V} blockers are commonly used to treat focal epilepsy whereby they reduce pathologic neuronal hyperexcitability to prevent the development of seizures or terminate a seizure state. However, approved Na_{V} blockers are not well tolerated, possibly due to an inability to selectively target hyperexcitable states.

### Background and Clinical Trial Rationale

There is a significant need for better tolerated Na_{V} blockers for patients with focal epilepsy. Compound 1 has the potential to be a safe and effective treatment for patients with this condition and has shown efficacy in rodent models of epilepsy. Currently available standard-of-care Na_{V} blockers used to treat epilepsy are limited by a low therapeutic index and a requirement to titrate to an efficacious concentration while managing tolerability. Significant class-related adverse effects are central nervous system (CNS)-related, including ataxia, drowsiness, and dizziness, which may be due to the excessive and long-lasting inhibition of peak I_{Na} resulting in an inability of existing Na_{V} blockers to selectivity reduce hyperexcitability while sparing normal brain function.

Preclinical data demonstrate that Compound 1 differentiates from approved Na_{V} blockers in several ways leading to an increased preclinical protective index (range between exposures that are efficacious in the maximal electroshock assay MES seizure model and exposures that reduce spontaneous movement in the spontaneous locomotor activity assay model). This increased protective index may predict greater clinical tolerability for Compound 1. Features of Compound 1 that may support this increased protective index are proposed to be an increase in the potency for persistent and peak I_{Na}, rapid I_{Na} inhibition kinetics, increase in activity-dependent inhibition of peak I_{Na}, and increased selectivity against non-Na_{V} mediated activity.

The safety, tolerability, pharmacokinetics (PK), and pharmacodynamics (PD) of Compound 1 are currently being evaluated in healthy volunteers in an ongoing first-in-human trial (see Example 12). Compound 1 was observed to be safe and generally well tolerated in healthy participants at all single doses up to 45 mg tested in Part A of the trial. All treatment-emergent adverse events (TEAEs) reported thus far have been mild in severity and resolved without administration of concomitant medication. No severe TEAEs were reported. The most frequently reported TEAEs were headache and fatigue. No deaths or TEAEs leading to study drug discontinuation have been reported. No safety findings were observed with regards to clinical laboratory test results, electrocardiograms (ECGs), and vital signs.

### Scientific Rationale for Clinical Trial Design

The assessment of potential new drugs for the treatment of epilepsy continues to be challenging. Epilepsy is characterized by different aetiologies and pathophysiological mechanisms. This leads to unpredictable electroencephalographic expressions and clinical manifestations for the disease. Clinical assessments that can be simply and reproducibly performed are useful in determining the potential of new treatments in development. Suppression of photoparoxysmal EEG response (PPR) has emerged over the past several decades as a valuable translation tool in early clinical development for the assessment of potential anti-seizure drugs (ASDs) with a variety of mechanisms of action.

Visual sensitive epilepsy is a reflex type of epilepsy; the epileptogenic reaction can be evoked systematically at any time in response to flashing lights.

Photosensitivity, defined as a generalized epileptiform reaction elicited by intermittent photonic stimulation (IPS), is found in approximately 5% of all epileptic patients. Although most prevalent in idiopathic generalized epilepsies, photosensitivity may also be present in other types of epilepsy and most notably in specific genetically determined syndromes, like Dravet syndrome.

Markedly visual sensitive patients are usually sensitive to IPS within clearly defined limits of flash frequency (mostly between 10-30 Hz). This photosensitivity range, the difference between the highest and lowest flash rates that consistently elicit a PPR, can be used as a quantitative measure of photosensitivity and, therefore, epileptogenicity. The photosensitivity range is related to the liability of visually induced seizures in daily life and is remarkably stable under controlled conditions. Previous studies show that the administration of marketed and experimental ASD, either in a single or repeated dose regimen, can have an impact on the PPR, diminishing or even abolishing the response to IPS. As measured by the EEG response to IPS, the pharmacodynamic effects are also not necessarily time-locked to the drug's pharmacokinetic profile, as was demonstrated in a single dose of valproic acid, levetiracetam and carisbamate. Combined with blood level monitoring, this study design offers information about the time of onset and the duration of the anti-epileptic action and some effects of tolerability of the drug.

Using a standardized, meticulously performed IPS procedure (short delivery of flashes, determination of threshold frequencies, for which the patient shows an epileptiform EEG response and simultaneous recording of the EEG and observation of the patient) brings the likelihood of provoking prominent clinical seizures to extremely low. Furthermore, IPS is a standard procedure in any routine EEG recording, like hyperventilation.

In summary, valuable preliminary information can be obtained using the PPR model. These studies are usually of short duration and are conducted in a limited number of patients under maximally standardized and controlled conditions. Positive results indicate target engagement and would support further clinical development of the drug that is investigated.

A single-blind, placebo-controlled, fixed-sequence design has been used by many others to successfully evaluate the IPS-induced PPR as a proof of principle for anti-epileptic drugs. This study design is preferred for 3 key reasons. First, this is the first in-patient study for Compound 1. Therefore, allowing it to only be blinded to the participant and the EEG reader provides for more diligent safety oversight during the Intervention Period. Second, each participant can serve as its own placebo control, which enables fewer participants to be exposed to study drug to achieve the study objectives. Reducing the total sample size allows for more efficient study conduct considering the rare prevalence of the patient population. Third, a PD effect can be observed after a limited number of participants are exposed to a single dose of an anti-epileptic drug. Therefore, if no PD effect is observed after administration of a lower dose of study drug to a limited number of participants, then escalation of the dose should be considered.

### Justification for Dose

A single dose will be sufficient to observe a suppression of the IPS-induced PPR.

Dose levels for this first-in-patient trial have been determined based on results from an ongoing first-in-human trial designed to investigate the safety, tolerability, PK, and PD of single ascending doses and multiple ascending doses of Compound 1 in healthy volunteers. All single doses evaluated to date (5 mg, 15 mg, and 45 mg) have been safe and generally well tolerated. At their peak, mean Compound 1 plasma concentrations for the 15 mg and 45 mg doses exceeded the estimated human equivalent exposure of the mouse MES EC₅₀, the concentration predicted to be required for modulation of sodium channels and the intended biological effect in patients. Based on the MES model, doses of 15 mg and 45 mg of Compound 1 are expected to produce maximum concentrations that approximate the EC₇₀ and EC₉₀, respectively. Given these data, the dose level of 15 mg in Part A is expected to be able to suppress the PPR response in participants. Further information is available in the investigator's brochure.

### Trial Objectives

The primary objective of this trial is to evaluate the pharmacodynamic effect of Compound 1 compared with placebo on the intermittent photic stimulation (IPS)-induced photoparoxysmal electroencephalogram response (IPS-induced PPR) in participants with epilepsy and IPS-induced PPR.

| | |
|---|---|
| Objective | Endpoint |

Primary

| | |
|---|---|
| To evaluate the pharmacodynamic effect of | Reduction or abolishment of the IPS- |
| Compound 1 compared with placebo on the | induced PPR |
| IPS-induced photoparoxysmal | |
| electroencephalogram response (IPS- | |
| induced PPR) in participants with | |
| epilepsy and IPS-induced PPR | |

Secondary

| | |
|---|---|
| To evaluate the pharmacokinetics of | Plasma concentrations of Compound 1 |
| Compound 1 in participants with epilepsy | Maximum observed concentration (Cₘₐₓ) |
| and IPS-induced PPR | Time to maximum observed |
| | concentration (tₘₐₓ) |
| | Area under the concentration-time curve |
| | from time zero to 24 hours (AUC₂₄) |
| To evaluate the tolerability and safety | Incidence and severity of adverse events |
| of Compound 1 compared to placebo in | (AEs) |
| participants with epilepsy and IPS-induced | Changes in vital sign measurements |
| PPR | Changes in clinical laboratory results |
| | Changes in electrocardiogram (ECG) |
| | parameters |

Exploratory

| | |
|---|---|
| To evaluate the pharmacodynamic effects of | Changes in qEEG biomarkers (absolute and |
| Compound 1 compared with placebo on | relative powers in the delta, theta, beta, |
| quantitative electroencephalography (qEEG) | alpha and gamma bands) compared to |
| measures in participants with epilepsy and | baseline |
| IPS-induced PPR | |

### Overall Design

This is a Phase 2, single-blind, placebo-controlled, fixed-sequence design trial to evaluate the photoparoxysmal EEG-response, safety, tolerability, and pharmacokinetics of Compound 1 in participants aged 18 to 65 years (inclusive) with epilepsy and IPS-induced PPR. The trial initially comprises 1 part (Part A), with 1 additional optional part (Part B). Both parts have identical design and will only differ in the dose of Compound 1.

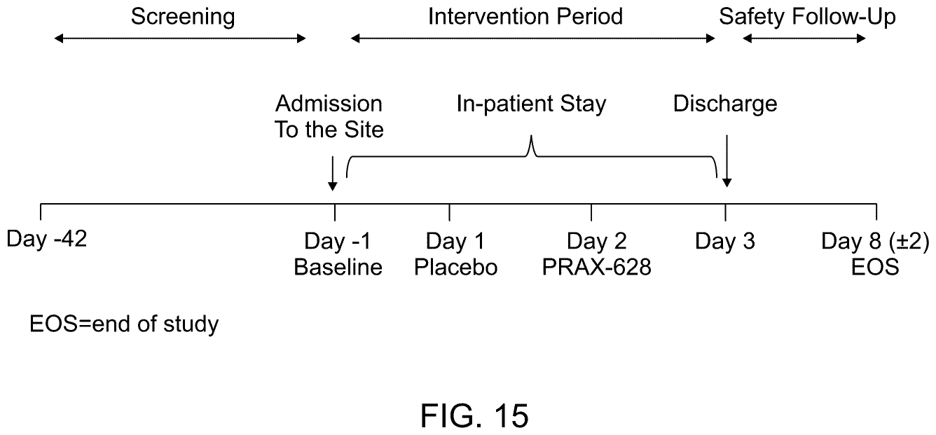

The trial consists of 3 periods: Screening/Baseline, Intervention, and Safety Follow-Up. FIG. 15 is an illustration of the dosing scheme for the trial.

### Screening/Baseline Period

The Screening period will be up to 42 days in duration (Day −42 to Day −2). Prior to any clinical trial procedures, participants will provide written informed consent to participate in the trial. Following confirmation of continued eligibility, participants will check in to the clinic at Baseline (Day −1; the day before study drug administration).

### Intervention Period

Participants will remain in the unit from Baseline (Day −1) to Discharge (Day 3). If participants experience any clinically significant AEs during their in-patient stay, they may remain in the clinical facility for further observation at the discretion of the principal investigator (PI), following consultation with the sponsor.

Participants with epilepsy and an IPS-induced PPR will receive a single dose of placebo on the morning of Day 1 and a single dose of Compound 1 on the morning of Day 2. On Days 1 and 2, IPS-induced PPR will be measured pre-dose and at 1, 2, 3, 4, 6, and 8 hours post-dose. The Day 3 assessment will occur 24 hours after the Day 2 dose. On Days 1 and 2, quantitative EEG (qEEG) will be performed predose and before the IPS assessment at 3 hours postdose. Blood collection for PK parameters will be collected 30 minutes after the assessment of IPS-induced PPR.

In Part A, up to 12 participants will receive 15 mg of Compound 1. The sponsor may recommend proceeding to an optional Part B if less than 75% of the participants cumulatively evaluated in Part A have demonstrated an insufficient reduction on PPR, namely a standardized photosensitivity range (SPR) reduced by less than 3 points in ≥1 eye conditions (eyes open, eye closure, and/or eyes closed) over ≥3 testing times within 1 day, compared to the range at the same time points on Day 1. The dose for Part B, if needed, will be established based on ongoing unblinded review of emerging data from Part A and results from the trial described in Example 12.

On every dosing day, safety assessments will include a neurological exam (including cranial nerves, coordination, and gait) and symptom-directed physical examination to be conducted pre-dose (within 2 hours before dosing) and 4 hours post-dose. Additionally, clinical laboratory parameters, including chemistry and hematology, will be evaluated on Baseline Day −1, pre-dose on Day 2, and at any time for all the other visits. Vital signs will be performed pre-dose (within 2 hours before dosing) and then 2, 4, 6, and 8 hours (±15 minutes) post-dose. ECG will be performed pre-dose (within 2 hours before dosing) and then 2, 4, and 6 hours (±15 minutes) post-dose. Participants will be discharged on Day 3 after a satisfactory safety review and completion of trial-related procedures. If a participant discontinues the Intervention Period early, an Early Termination (ET) visit will be conducted.

### Safety Follow-Up Period

A Safety Follow-Up visit will be conducted on Day 8 (±2 days).

### Number of Participants

This trial will enroll up to 12 participants (up to 12 participants total across both Part A and the optional Part B). Participants who withdraw or are withdrawn from the study prior to completion of the nominal clinical conduct for reasons other than the occurrence of a serious adverse event (SAE) may be replaced.

### Clinical Trial Intervention

For Part A and Part B, each study participant will receive placebo capsule(s) on Day 1 and Compound 1 capsule(s) on Day 2. On each dosing day, a single dose of Compound 1 (15 mg for Part A, to be determined for Part B) or matching placebo will be administered orally (fasted) and provided in single dose containers to the participants.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all constructs, methods, and/or component features, steps, elements, or other aspects thereof can be used in various combinations.

Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure also includes embodiments in which more than one, or the entire group members are present in, employed in, or otherwise relevant to a given product or process. Furthermore, it is to be understood that the disclosure encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, (e.g., in Markush group or similar format) it is to be understood that each subgroup of the elements is also disclosed, and any element(s) can be removed from the group. In general, where embodiments or aspects of the disclosure, is/are referred to as comprising particular elements, features, etc., certain embodiments or aspects consist, or consist essentially of, such elements, features, etc. For purposes of simplicity those embodiments have not in every case been specifically set forth in so many words herein. It should also be understood that any embodiment or aspect of the disclosure can be explicitly excluded from the claims, regardless of whether the specific exclusion is recited in the specification.

All patents, patent applications, websites, other publications or documents, accession numbers and the like cited herein are incorporated by reference in their entirety for all purposes to the same extent as if each individual item were specifically and individually indicated to be so incorporated by reference.

We claim:

1. A method of treating a condition relating to aberrant function of a sodium ion channel in a subject in need thereof, said method comprising administering to said subject Compound 1, or a pharmaceutically acceptable salt thereof, at a dose of about 10 mg to about 60 mg;
wherein Compound 1 is of the following structural formula: 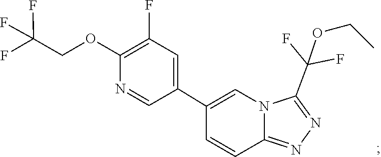 [SMILES: CCOC(F)(F)c1nnc2ccc(-c3cnc(OCC(F)(F)F)c(F)c3)cn12]
wherein the condition is generalized epilepsy or focal epilepsy; and
wherein the subject is a human.

2. The method of claim 1, wherein the condition is generalized epilepsy.

3. The method of claim 1, wherein the condition is focal epilepsy.

4. The method of claim 1, wherein administration of Compound 1, or a pharmaceutically acceptable salt thereof, results in suppression of photoparoxysmal (PPR) response.

5. The method of claim 1, wherein Compound 1, or a pharmaceutically acceptable salt thereof, is administered at a dose of about 15 mg, about 20 mg, about 25 mg, about 30 mg, about 35 mg, about 40 mg, about 45 mg, about 50 mg, about 55 mg or about 60 mg.

6. The method of claim 5, wherein Compound 1, or a pharmaceutically acceptable salt thereof, is administered at a dose of about 15 mg.

7. The method of claim 5, wherein Compound 1, or a pharmaceutically acceptable salt thereof, is administered at a dose of about 45 mg.

8. The method of claim 1, wherein Compound 1, or a pharmaceutically acceptable salt thereof, is administered orally.

9. The method of claim 1, wherein Compound 1, or a pharmaceutically acceptable salt thereof, is administered once daily.

10. The method of claim 1, wherein the subject is a fetus or a pediatric subject.

11. The method of claim 10, wherein the pediatric subject is an infant, a child or an adolescent.

12. The method of claim 1, wherein the subject is an adult subject.

13. The method of claim 12, wherein the subject is a young adult, a middle-aged adult or a senior adult.
